(19) United States
(12) Patent Application Publication
(10) Pub. No.: US 2009/0203017 A1
(43) Pub. Date: Aug. 13, 2009

(54) Use of Nucleic Acid Probes to Detect Nucleotide Sequences of Interest in a Sample

(75) Inventors: ELLIOTT P. DAWSON, MURFREESBORO, TN (US); STEVEN J. SIMMONS, UNIONVILLE, TN (US); LORI J. RAY-COX, MURFREESBORO, TN (US); JENNIFER M. BAKER, NASHVILLE, TN (US); KRISTIE E. WOMBLE, FRANKLIN, TN (US); JUDITH MADDEN, VALENCIA, CA (US); SUBRAMANI SELLAPPAN, AURORA, IL (US); ANDREW HEARN, CHICAGO, IL (US); SAL SEMINARA, CHICAGO, IL (US)

Correspondence Address: LOEB & LOEB, LLP, 321 NORTH CLARK, SUITE 2300, CHICAGO, IL, 60654-4746, US

(73) Assignees: BLOVENTURES, INC., MURFREESBORO, TN (US); Celsis International plc, Chicago, IL (US)

(21) Appl. No.: 12/359,263

(22) Filed: Jan. 23, 2009

Related U.S. Application Data
(60) Provisional application No. 61/023,348, filed on Jan. 24, 2008.

(51) Int. Cl. C12Q 1/68

(52) U.S. Cl. 435/6

(57) ABSTRACT

The invention relates to methods for the determination and detection of nucleic acids sequences in a sample. The nucleic acid may be RNA or DNA or both. The invention also relates to methods for the determination of the presence and species of various microorganisms in a sample. We have also identified a set of oligonucleotide nucleic acid sequences within the rRNAs of Gram-negative organisms that facilitates both the broad identification of Gram-negative organisms as a class when used as a pool, or in combination, for example in a hybridization assay. This set of oligonucleotides may detect sequences that are indicative of the presence of organisms of the broad class of Gram-negative organisms while exhibiting little or no false identification of Gram-positive organisms, and fungi, or other microorganisms. The assay includes concurrent incubation with at least one nucleotide sequence of interest, at least one nucleic acid probe, a fluorosurfactant, and a nuclease. The assay may further be employed to detect the presence of bacteria, fungi, or other microorganisms by use of additional specific probes, or to detect and/or identify target nucleic acid sequences in a sample. Further, the invention also relates to methods of reducing non-specific binding and facilitating complex formation in a binding assay. The binding assay may be, but is not limited to, a nucleic acid hybridization assay or an immunoassay. The invention also relates to methods of detection that employ at least one target of interest, which may be a nucleotide sequence, at least one probe, which may be a nucleic acid probe and a nuclease.

This application claims benefit of priority to U.S. Provisional Patent Application No. 61/023,348, filed on Jan. 24, 2008, which application is incorporated by reference in its entirety.

### FIELD OF THE INVENTION

The invention relates to methods for the determination and detection of nucleic acids sequences of interest in a sample. The nucleic acid may be RNA or DNA or both. The invention also relates to methods for the determination of the presence and species of various microorganisms in a sample. We have also identified a set of oligonucleotide nucleic acid sequences within the rRNAs of Gram-negative organisms that facilitates both the broad identification of Gram-negative organisms as a class when used as a pool or in combination, for example in a hybridization assay. This set of oligonucleotides may detect sequences that are indicative of the presence of organisms of the broad class, of Gram-negative organisms while exhibiting little or no false identification of Gram-positive organisms, and fungi, or other microorganisms. The assay may further be employed to detect the presence of bacteria, fungi, or other microorganisms by use of additional specific probes, as well as to detect and/or identify target nucleic acid sequences in a sample.

The invention also relates to an assay that includes concurrent incubation with at least one nucleotide sequence of interest, at least one nucleic acid probe labeled with a detectable label, an optional fluorosurfactant, and a nuclease. The assay may further be employed to detect the presence of bacteria, fungi, or other microorganisms, as well as naked nucleotide sequences, by use of additional specific probes, or to detect and/or identify target nucleic acid sequences in a sample. Further, the invention relates to methods of reducing non-specific binding and facilitating complex formation in a binding assay. The binding assay may be, but is not limited to, an immunoassay, a microfluidic assay, passivation of vessels, cell culture, or a nucleic acid hybridization assay. The invention also relates to methods of detection that employ at least one target of interest, which may be a nucleotide sequence, at least one probe, (e.g., a nucleic acid probe), and a nuclease. Other molecules and compounds that can be detected include, but are not limited to, proteins, peptides, small chemical molecules, carbohydrates, lipopolysaccharides, polysaccharides, and lipids. The invention additionally relates to a kit for carrying out such assays.

### BACKGROUND OF THE INVENTION

Each of the cells of all life forms, except viruses, contain ribosomes and therefore ribosomal RNA. A ribosome contains three separate single strand RNA molecules, namely, a large molecule, a medium sized molecule, and a small molecule. The two larger rRNA molecules vary by size in different organisms. Ribosomal RNA is a direct gene product and is coded for by the rRNA gene. The DNA sequence of the gene is used as a template to synthesize rRNA molecules. A separate gene exists for each of the ribosomal RNA subunits. Multiple rRNA genes exist in most organisms, with many higher organisms containing both nuclear and mitochondrial rRNA genes. Plants and certain other forms contain nuclear, mitochondrial and chloroplast rRNA genes. For simplicity, the three separate rRNA genes will be referred to as the rRNA gene.

Numerous ribosomes are present in all cells of all life forms. About 85-90 percent of the total RNA in a typical cell is rRNA. A bacteria such as E. coli contains about 10⁴ ribosomes per cell, while a mammalian liver cell contains about 5×10⁶ ribosomes per cell. Since each ribosome contains one of each rRNA subunit, the bacterial cell and mammalian cell contains 10⁴ and 5×10⁶, respectively, of each rRNA subunit.

Ribonucleic acids, other than ribosomal RNA, especially messenger RNAs are highly useful in determining identity, metabolic or disease state or the presence of active viral infections both from RNA or DNA viruses. Determination of either transcript identification, level of expression or both can be of immense benefit in diagnostics and life sciences research as well in various quality control assays. Measurement of additional forms of RNAs such as microRNAs can likewise be highly beneficial to diagnostic determination, especially in cancer.

Nucleic acid hybridization, a procedure well-known in the art, has been used to specifically detect extremely small or large quantities of a particular nucleic acid sequence, even in the presence of a very large excess of non-related sequences. Many prior art uses of nucleic acid hybridization are found in publications involving molecular genetics of cells and viruses; genetic expression of cells and viruses; genetic analysis of life forms; evolution and taxonomy or organisms and nucleic acid sequences; molecular mechanisms of disease processes; and diagnostic methods for specific purposes, including the detection of viruses and bacteria in cells and organisms.

Probably the best characterized and most studied gene and gene product are the rRNA gene and rRNA. The prior art includes use of hybridization of rRNA and ribosomal genes in genetic analysis, as well as the evolutionary and taxonomic classification of organisms and ribosomal gene sequences. Genetic analysis includes, for example, the determination of the numbers of ribosomal RNA genes in various organisms, the similarity between the multiple ribosomal RNA genes which are present in cells, and the rate and extent of synthesis of rRNA in cells; and the factors which control them. Evolutionary and taxonomic studies often involve comparing the rRNA gene base sequence from related and widely different organisms.

It is known that the ribosomal RNA gene nucleotide sequence is at least partially similar in widely-different organisms. For example, the DNA of E. coli bacterial ribosomal RNA genes hybridizes with rRNA from plants, mammals, and a wide variety of bacterial species. The fraction of the E. coli gene which hybridizes to these other species varies with the degree of relatedness of the organisms. Virtually all of the rRNA gene sequence hybridizes to rRNA from closely-related species, but hybridizes less well to rRNA from distantly related species.

The sensitivity and ease of detection of specific groups of organisms by utilizing probes specific for the rRNA of that group is greatly enhanced by the large number of both rRNA molecules which are present in each cell as single-stranded nucleic acid molecules. However, though the rRNA is a single-stranded molecule, it has extraordinary, highly convoluted secondary and tertiary structures, which can make access of probes to certain segments extremely difficult. Even denaturation in solution offers only partial relief from a snap-back tendency of the rRNA to reform these secondary and tertiary structures. This tendency rapidly occurs because the snap-back is an intramolecular refolding and is not diffusion limited. One may overcome this problem by denaturing the RNA and immobilizing it in its denatured state using techniques such as adsorption to nitrocellulose or other supports, or using probes having a higher melting temperature (Tm) than the target segment's internal complement, which may obstruct the self annealing of the molecule.

Besides, rRNA probes specific for other classes of cell nucleic acids, besides rRNA, may be used to specifically detect, identify, and quantitate specific groups of organisms or cells by nucleic acid hybridization. For example, rRNA is synthesized in the bacteria E. coli as a precursor molecule about 6000 bases long. This precursor molecule is then processed to yield both rRNA subunits (totaling about 4500 bases), which are incorporated into ribosomes, and some extra RNA sequences (1500 bases in total), which are discarded.

A well-known amplification method is the polymerase chain reaction (PCR). In PGR, a characteristic piece of the particular nucleotide sequence of interest is amplified with specific primers. If the primer finds its target site, a sequence of the genetic material undergoes a million-fold proliferation.

During the PCR process, the DNA generated is used as a template for replication. This sets in motion a chain reaction in which the DNA template is exponentially amplified. PCR can amplify a single or few copies of a piece of DNA by several orders of magnitude, generating millions or more copies of the DNA piece. PCR can be extensively modified to perform a wide array of genetic manipulations.

Almost all PCR applications employ a heat-stable DNA polymerase. One example is Taq polymerase, an enzyme originally isolated from the bacterium Thermus aquaticus. This DNA polymerase enzymatically assembles a new DNA strand from DNA building blocks, the nucleotides, by using single-stranded DNA as a template and DNA oligonucleotides (also called DNA primers), which are required for initiation of DNA synthesis. The vast majority of PCR methods use thermal cycling, i.e., alternately heating and cooling the PCR sample to a defined series of temperature steps. These thermal cycling steps are necessary to physically separate the strands at high temperatures in a DNA double helix (DNA melting) used as the template during DNA synthesis at lower temperatures by the DNA polymerase to selectively amplify the target DNA. The selectivity of PCR results from the use of primers that are complementary to the DNA region targeted for amplification under specific thermal cycling conditions.

A PCR reaction usually consists of a series of 20 to 40 repeated temperature changes called cycles. Each cycle typically consists of 2-3 discrete temperature steps. Most commonly, PCR amplifications are carried out with cycles that have three temperature steps. The cycling is often preceded by a single temperature step (called hold) at a high temperature (>90° C.), and followed by one hold at the end for final product, extension or brief storage. The temperatures used and the length of time they are applied in each cycle depend on a variety of parameters. These include the enzyme used for DNA synthesis, the concentration of divalent ions and dNTPs in the reaction, and the melting temperature (Tm) of the primers.

The initiation step consists of heating the reaction to a temperature of about 94-96° C. (or 98° C. if extremely thermostable polymerases are used), which is held for 1-9 minutes. This step is only required for DNA polymerases that require heat activation by hot-start PCR.

The denaturation step is the first regular cycling event and consists of heating the reaction to 94-98° C. for 20-30 seconds. This step causes melting of DNA template and primers by disrupting the hydrogen bonds between complementary bases of the DNA strands, yielding single strands of DNA.

Next, during the annealing step reaction, the temperature is lowered to about 50-65° C. for 20-40 seconds, which allows annealing of the primers to the single-stranded DNA template. Typically the annealing temperature is about 3-5 degrees Celsius below the Tm of the primers used. Stable DNA-DNA hydrogen bonds are only formed when the primer sequence very closely matches the template sequence. The polymerase binds to the primer-template hybrid and begins DNA synthesis.

The extension/elongation step has a temperature that depends on the DNA polymerase used. For example, Taq polymerase has its optimum activity temperature at about 75-80° C., and commonly a temperature of 72° C. is used with this enzyme. At this step the DNA polymerase synthesizes a new DNA strand complementary to the DNA template strand by adding dNTPs that are complementary to the template in 5′ to 3* direction, condensing the 5′-phosphate group of the dNTPs with the 3′-hydroxyl group at the end of the nascent (extending) DNA strand. The extension time depends both on the DNA polymerase used and on the length of the DNA fragment to be amplified. At its optimum temperature, the DNA polymerase will typically polymerize a thousand bases per minute. Under optimum conditions, i.e., if there are no limitations due to limiting substrates or reagents, at each extension step, the amount of DNA target is doubled, leading to exponential (geometric) amplification of the specific DNA fragment.

The final elongation is occasionally performed at a temperature of 70-74° C. for 5-15 minutes after the last PCR cycle to ensure that any remaining single-stranded. DNA is fully extended. A final hold at 4-15° C. may be employed for short-term storage of the reaction.

A qualitative evaluation may be made in the above analysis using, for example, an agarose gel that separates DNA fragments. In the most simple ease, this evaluation provides the information that the target sites for the primers were present in the analysis sample.

Another development of the PCR technique is quantitative PCR, which seeks to establish a correlation between the quantity of microorganisms present and the quantity of amplified DNA. Additionally, reverse transcriptase PCR allows the detection of RNA species within a sample and permits the use of the detected RNAs to serve as a proxy for the distinction between live and dead organisms or free DNA. However, DNA present in the sample prior to amplification must be rigorously eliminated to prevent false positives so that only RNAs present in the sample give rise to amplification products.

Advantages of PCR include its high specificity and the relatively short time it takes to perform. Major disadvantages are its high susceptibility to contamination and the resulting false-positive results, the above-mentioned impossibility of distinguishing between living and dead cells or naked DNA unless reverse transcriptase PCR is performed, and finally, the danger of false-negative results due to the presence of inhibitory substances. Many of these disadvantages can be overcome by adaptation of suitable laboratory practices or protocol designs which may add non-trivial increases of costs, equipment, facility requirements, time, and expertise for their implementation.

Using in situ hybridization with fluorescence-marked oligonucleotides is another useful process, and was developed at the beginning of the 1990's. This process has been successfully used in many environmental samples (Amann et al., Fluorescent-oligonucleotide probing of whole cells for determinative, phylogenetic, and environmental studies in microbiology, 172(2) J. BACTERIOL. 762-770 (1990)), and this process is commonly known as “FISH” (fluorescence in situ hybridization). The FISH technique is a tool for specifically detecting microorganisms in a sample and with specificity and relies on the premise that the ribosomal ribonucleic acids (rRNAs) occurring in every cell have both highly preserved and variable sequences, i.e., genus- or even species-specific. Complementary oligonucleotides may be produced against these sequence domains and may be additionally provided with a detectable marker, which enables the identification of microorganism species, genera, or groups. The FISH method is the only commonly-known method which provides a distortion-free representation of the actual in situ conditions of the bibcocnosis, where non-cultivated and un-described microorganisms may be identified.

In FISH, probes penetrate into the cells, present in the analysis sample and bind to their target sequence within the cell, enabling detection of the cell through the marking of the probes. FISH may also be used to identify microorganisms that are difficult to detect by traditional cultivation, which enables a bacterial population to be detected in many samples. FISH may be used to detect microorganisms more quickly than by cultivation.

FISH can also be used determine certain morphologies by visualization of the cells and/or tissues. False negative results due to the presence of inhibitory substances can be ruled out, as much as false-positive results attributable to contaminations.

Many references relating to molecular biology have been published over the years. These references disclose microbiological culture protocols; structure and function of nucleic acids, such as rRNA; methods relating to the identification of microorganisms; nucleic acid hybridization techniques; and nucleic acid probe determination

The following references relate to protocols and methods for culturing bacteria and other microorganisms and the extraction of nucleic acids: Brian W. Bainbridge, Microbial techniques for molecular biology: bacteria, and phages in ESSENTIAL MOLECULAR BIOLOGY: A PRACTICAL APPROACH xv-xvii, 21-54 (T. A. Brown, ed. 2000); Laura G. Leff et al., Comparison of Methods of DNA Extraction from Stream Sediments, 61(3) APPL. ENVIRON. MICROBIOL. 1141-1143 (1995); Yu-Li Tsai & Betty H. Olson, Rapid Method for Direct Extraction of DNA from Soil and Sediments, 57(4) APPL. ENVIRON. MICROBIOL. 1070-1074 (1991); TECHNOTE 302 MOLECULAR BIOLOGY (Bangs Laboratories, Inc. 2002).

The following references relate to studies on the structure and function of ribosomal RNA: Sebastian Behrens et al., In Situ Accessibility of Small-Subunit rRNA of Members of the Domains Bacteria, Archaea, and Eucarya to Cy3-Labeled Oligonucleotide Probes, 69(3) APPL. ENVIRON. MICROBIOL. 1748-1758 (2003); Soumitesh Chakravorty et al., A detailed analysis of 16S ribosomal RNA gene segments for the diagnosis of pathogenic bacteria, 69(2) J. MICROBIOL. METHODS 330-339 (2007); Darrell P. Chandler et al., Sequence versus Structure for the Direct Detection of 16S rRNA on Planar Oligonucleotide Microarrays, 69(5) APPL. ENVIRON. MICROBIOL. 2950-2958 (2003); Bernhard M. Fuchs et al., Unlabeled Helper Oligonucleotides Increase the In Situ Accessibility to 16S RNA of Fluorescently Labeled Oligonucleotide Probes, 66(8) APPL. ENVIRON. MICROBIOL. 3603-3607 (2000); Danielle A. M. Konings & Robin R. Gutell, A comparison of thermodynamic foldings with comparatively derived structures of 16S and 16S-like rRNAs, 1 RNA 559-574 (1995); Harry F. Noller et al., Studies on the structure and function of 16S ribosomal RNA using structure-specific chemical probes, 8(3 & 4) PROC. INT. SYMP. BIOMOL. STRUCT. INTERACTIONS, SUPPL. J. BIOSCI. 747-755 (1985); Alex Pozhitkov et al., Tests of rRNA hybridization to microarrays suggest that hybridization characteristics of oligonucleotide probes for species discrimination cannot be predicted, 34(9) NUCLEIC ACIDS RES. e66 (2006); Miguel Ángel Reyes-López et al., Fingerprinting of prokaryotic 16S rRNA genes using oligodeoxyribonucleotide microarrays and virtual hybridization, 31(2) NUCLEIC ACIDS RES. 779-789 (2003); Achim Schmalcnberger et al., Effect of Primers Hybridizing to Different Evolutionarily Conserved Regions of the Small-Subunit rRNA Gene in PCR-Based Microbial Community Analyses and Genetic Profiling, 67(8) APPL. ENVIRON. MICROBIOL. 3557-3563 (2001); S.-G. Tao et al., Room-Temperature Hybridization of Target DNA with Microarrays in Concentrated Solutions of Guanidine Thiocyanate, 34(6) BIOTECHNIQUES 1261, 1262 (2003); C. R. Woese et al., Secondary structure model for bacterial 16S ribosomal RNA: phylogenetic, enzymatic and chemical evidence, 8(10) NUCLEIC ACIDS RES. 2275-2293 (1980); L. Safak Yilmaz & Daniel R. Noguera, Mechanistic Approach to the Problem of Hybridization Efficiency in Fluorescent In Situ Hybridization, 70(12) APPL. ENVIRON. MICROBIOL. 7126-7139 (2004); L. Safak Yilmaz et al., Making All Parts of the 16S rRNA of Escherichia coli Accessible In Situ to Single DNA Oligonucleotides, 72(1) APPL. ENVIRON. MICROBIOL. 733-744 (2006).

The following references relate to the identification and differentiation of bacteria and other microorganisms: Sven Klaschik et al., Real-Time PCR for Detection and Differentiation of Gram-Positive and Gram-Negative Bacteria, 40(11) J. CLIN. MICROBIOL. 4304-4307 (2002); Elizabeth m. Marlowe et al., Application of an rRNA Probe Matrix for Rapid Identification of Bacteria and Fungi from Routine Blood Cultures, 41 (11) J. CLIN. MICROBIOL. 5127-5133 (2003).

The following references relate to the use of hybridization techniques in molecular biology: Francois Coutlce et al., Quantitative Detection of Messenger RNA by Solution Hybridization and Enzyme Immunoassay, 265(20) J. BIOL. CHEM. 11601-11604 (1990); Gary K. McMaster & Gordon G. Carmichael, Analysis of single- and double-stranded nucleic acids on polyacrylamide and agarose gels by using glyoxal and acridine orange, 74(11) PROC. NATL. ACAD. SCI. USA 4835-4838 (1977); Southern Hybridization—Genomic ES Cell DNA.

The following references relate to ribosomal RNA probe accessibility using ARB software (ARB is derived from the Latin word arbor, or tree): Yadhu Kumar et al., Graphical representation of ribosomal RNA probe accessibility data using ARB software package, 6 BMC BIOINFORMATICS 61 (2005); Yadhu Kumar et al., Evaluation of sequence alignments and oligonucleotide probes with respect to three-dimensional structure of ribosomal RNA using ARB software package, 7 BMC BIOINFORMATICS 240 (2006); Yadhu Kumar et al., presentation entitled Visualization of Probe Accessibility of Ribosomal RNA using ARB Software.

The following references relate to probes and primers for 16S rRNA: K. Greisen et al., PCR Primers and Probes for the 16S rRNA Gene of Most Species of Pathogenic Bacteria, Including Bacteria Found in Cerebrospinal Fluid, 32(2) J. CLIN. MICROBIOL. 335-351 (1994); Philip Hugenholtz et al., Design and Evaluation of 16S rRNA-Targeted Oligonucleotide Probes for Fluorescence In Situ Hybridization, 179 METHODS. MOL. BIOL. 29-42 (2002); Christopher Lay et al., Design and validation of 16S rRNA probes to enumerate members of the Clostridium leptum subgroup in human faecal microbiota, 7(7) ENVIRON. MICROBIOL. 933-946 (2005).

Nuclease protection assays represent another method employed for the detection of nucleic acids, especially RNAs. Most frequently, nuclease protection assays employ nucleases which digest single-stranded nucleotide sequences usually with a specificity for either DNA or RNA substrates. These nucleases typically show substantially diminished or no activity toward double-stranded forms of their respective substrate nucleic acids or chimeric double-stranded nucleic acids, i.e., double strands comprised of RNA:RNA, DNA:DNA, or RNA:DNA. In particular, ribonuclease protection assays enable the identification and characterization of RNA species including transcripts, exon/intron boundaries, and the like. Ribonuclease protection assays usually rely on hybridization between RNA probes and target RNAs, and digestion of non-hybridized RNAs by the action of an RNase, usually RNase A, RNaseT1, RNase I, or combinations of these RNases. The reactions are performed by combining the probe RNA and its target RNA, followed by their denaturation and subsequent annealing to yield a double-stranded RNA complex. This probe/target complex is treated with RNase to digest any non-hybridized segments, unhybridized probes, or other RNA molecules in the sample. Only the undigested double-stranded probe/target RNA segments should typically survive the nuclease digestion, which are subsequently analyzed by their detection.

Despite the number of research activities in these fields, methods for the isolation and detection of DNA or RNA from or within a number of different specimens—such as various tissues of plants, animals, and microbial organisms—often require sophisticated laboratories, expensive equipment, and well-trained, highly-educated personnel for reliable performance. Such methods also require sophisticated and subjective analytical techniques, like Sanger sequencing, PCR, qRT-PCR and RT-PCR, or FISH and array hybridization.

In addition, highly purified total nucleic acids, DNA, or RNA, along with carefully and precisely controlled conditions of temperature and time, are often required for nucleic acid isolation, nucleic acid detection, or both. When considered in their entirety, nucleic acid isolation and analysis times can be lengthy and usually require 3-6 hours from start to finish. In fact, overnight hybridizations are common for array-based methods. Moreover, where washing is required, strict temperature control is typically employed to provide stringency, especially in hybridization assays.

In any assay, background and non-specific signal can contribute to spurious signal and place inherent limits on assay sensitivity. Consequently, a large number of reagents and techniques have been developed to overcome and reduce background signal in assays. Yet, there is a need for further improvements to reduce signals arising from non-specific sources, because each assay system has characteristics which contribute to background or non-specific signals, and existing methods inadequately address this problem. Existing methods such as immunoassays or nucleic acid hybridization assays could derive benefit from improved sensitivity resulting from a reduction in background and/or non-specific signals.

Thus, a need exists for more sensitive and easier-to-use assays employing improved isolation methods, hybridization reagents and conditions for those methods, and oligonucleotides that may be used as nucleic acid probes, as well as sensitive and easy-to-use assays. A need also exists for assays that avoid using sophisticated instrumentation, extensively purified nucleic acids, or highly educated personnel and facilities to perform such assays. More particularly, a need exists for probes and assays used in research diagnostics, and for detecting microorganisms commonly in contact with human beings and/or animals, which are often found in foods, wastewaters, pharmaceutical and personal care products, and in the environment.

### SUMMARY OF THE INVENTION

The present invention relates to the finding that when, at least one nuclease is used in the presence of a nucleic acid probe and a target nucleic acid, the probe and target nucleic acid will readily form a detectable probe-target complex. While in the absence of the nuclease, no detectable probe:target complex will be formed. Such complexes will form considerably slower than those formed in the presence of the nuclease, or the formation of the complexes may require the use of precisely dictated conditions relating to temperature and solvents. Additionally, we have found that certain fluorosurfactants can reduce signals arising from non-specific binding of assay components (background). This reduction can improve assay sensitivity, especially in highly sensitive assays, for example, those employing chemiluminescence as modes of detection. The use of either one or both of these findings can improve nucleic acid hybridization assays. Several of these embodiments are described in more detail below.

The invention relates to a method of detecting the presence of at least one nucleotide sequence in a sample comprising providing a sample potentially containing at least one nucleotide sequence of interest; creating a mixture by combining the sample or the sequence of interest, at least one nucleic acid probe labeled with a detectable label, and a nuclease capable of degrading the sequence of interest; wherein the nuclease is added to the sample or the sequence of interest before or concurrently with adding the probe; and wherein a complex forms between the sequence of interest and the probe; and measuring the level of the detectable label in the complex, wherein the presence Of the detectable label in the complex indicates the presence of the sequence of interest.

The invention also relates to a method of detecting the presence of at least one nucleotide sequence in a sample comprising providing a sample potentially containing at least one nucleotide sequence of interest; creating a mixture by combining the sample or the sequence of interest, and a combination of at least one nucleic acid probe labeled with a detectable label, and a nuclease capable of degrading the sequence of interest; and wherein a complex forms between the sequence of interest and the probe; and measuring the level of the detectable label in the complex, wherein the presence of the detectable label in the complex indicates the presence of the sequence of interest.

The invention further relates to a method of detecting the presence of at least one nucleotide sequence in a sample comprising providing a sample potentially containing at least one nucleotide sequence of interest; creating a mixture by combining the sample or the sequence of interest, at least one nucleic acid probe, labeled with a detectable label, and a nuclease capable of degrading the sequence of interest; wherein the probe is added to the sample or the sequence of interest within a selected time period; wherein a complex forms between the sequence of interest and the probe; and measuring the level of the detectable label in the complex, wherein the presence of the detectable label in the complex indicates the presence of the sequence of interest.

The invention also relates to a method of detecting the presence of at least one nucleotide sequence in a sample comprising providing a sample potentially containing at least one nucleotide sequence of interest; creating a mixture by combining the sample or the sequence of interest, at least one nucleic acid probe labeled with a detectable label, and a nuclease capable of degrading the sequence of interest; wherein the nuclease is added to the sample or the sequence of interest and the probe before the sequence of interest hybridizes to the probe, resulting in a selected percentage of hybridization; wherein a complex forms between the sequence of interest and the probe; and measuring the level of the detectable label in the complex, wherein the presence of the detectable label in the complex indicates the presence of the sequence of interest.

The invention relates to a method of detecting the presence of at least one nucleotide sequence in a sample comprising providing a sample potentially containing at least one nucleotide sequence of interest; creating a mixture by combining the sample or the sequence of interest, at least one nucleic acid probe labeled with a detectable label, and a nuclease capable of degrading the sequence of interest; and at least one fluorosurfactant; wherein a complex forms between the sequence of interest and the probe; and measuring the level of the detectable label in the complex, wherein the presence of the detectable label in the complex indicates the presence of the sequence of interest.

The invention also relates to a kit for detecting the presence of at least one nucleotide sequence in a sample comprising at least one nucleotide probe labeled with a detectable label; a fluorosurfactant; and a nuclease capable of degrading a nucleotide sequence of interest.

The kit of the invention may also comprise instructions for using the kit. The kit may further comprise a reagent substrate. The kit may also include a lysis/extraction buffer and/or a wash buffer. Moreover, the kits of the invention, may be used for any of the methods for the detection of at least one nucleotide sequence of interest in a sample.

The invention also relates to a method of detecting the presence of a target of interest in a sample comprising providing a sample potentially containing a target of interest; creating a mixture by combining the sample or the target of interest; at least one probe labeled with a detectable label; and at least one fluorosurfactant; wherein a complex forms between the target of interest and the probe; and measuring the level of the detectable label in the complex, wherein the presence of the detectable label in the complex indicates the presence of the target of interest. The target of interest may be selected from the group consisting of proteins, peptides, small chemical molecules, carbohydrates, lipopolysaccharides, polysaccharides, and lipids.

The invention further relates to a method of reducing non-specific binding in an application comprising adding at least one fluorosurfactant to a buffer; and performing the application; wherein the presence of the at least one fluorosurfactant results in the reduction of non-specific binding on a surface. The application may be, but is not limited to, an immunoassay, a microfluidic assay, passivation of a vessel surface, or cell or tissue culture.

The present invention has the advantages of not requiring purified, isolated nucleic acids, along with a concurrent use of the enzyme RNase A, or another nuclease, in the presence of sample RNA (when RNA is the target) and in the presence of at least one nucleic acid probe. In addition, RNase A may be used to help reduce non-specific binding when sample total nucleic acids are used and DNA is the target. In addition, the use of RNase A or other nucleases to afford better access of probes to their respective targets is not only novel, but also applicable to other protocols, such as FISH or microarray assays. Furthermore, the assay of the present invention does not require any nucleic acid denaturing components, such as heat or chaotropic salts (normally required to isolate nucleic acids), or denaturation of the RNA in the sample prior to Or during an assay for RNA. Other nucleases that may be used are RNase T1, RNase I, and S1 nuclease.

The present invention also relates to a method of detecting the presence of a nucleotide sequence of interest in a sample comprising the steps of providing a sample potentially containing a nucleotide sequence(s) of interest to be analyzed; extracting the nucleotide sequence(s) from the sample; incubating the extracted nucleotide sequence(s) of interest with at least one nucleic acid probe under hybridization conditions so that the at least one nucleic acid probe hybridizes to the extracted nucleotide sequence(s) of interest to form a nucleotide sequence(s) of interest-probe complex, wherein the at least one nucleic acid probe is labeled with a detectable label; washing the nucleotide sequence(s) of interest-probe complex; adding a reagent substrate to the nucleotide sequence(s) of interest-probe complex; and measuring the level of hybridization via the detectable label, wherein detection of the detectable label indicates the presence of the nucleotide sequence(s) of interest.

Another embodiment of the invention relates to a method of differentiating Gram-negative bacteria from Gram-positive bacteria comprising the steps of providing a sample potentially containing microorganisms comprised of a nucleic sequence of interest to be analyzed; lysing the microorganisms; extracting, the nucleotide sequence of interest from the microorganisms; incubating the extracted nucleotide sequence of interest with at least one nucleic acid probe under hybridization conditions so that the at least one nucleic acid probe hybridizes to the extracted nucleotide sequence of interest to form a nucleotide sequence of interest-probe complex, wherein the at least one nucleic acid probe is labeled with a detectable label; washing the nucleotide sequence of interest-probe complex with a wash solution; adding a reagent substrate to the washed nucleotide sequence of interest-probe complex; and measuring the level of hybridization via the detectable label, wherein detection of the detectable label indicates the presence of a Gram-negative microorganism.

The present invention also relates to a method of detecting the presence of a nucleic acid in a sample comprising the steps of providing a cell sample potentially containing RNA to be analyzed; lysing the cells; extracting the RNA from the cells; incubating the extracted RNA with at least one nucleic acid probe under hybridization conditions so that the at least one nucleic acid probe hybridizes to the extracted RNA to form an RNA-probe complex, wherein the at least one nucleic acid probe is labeled with a detectable label; washing the RNA-probe complex with a wash solution; adding a reagent substrate to the washed RNA-probe complex; and measuring the level of hybridization via the detectable label, wherein detection of the detectable label indicates the presence of a RNA and identifying the cellular identity of the RNA. In addition, microorganisms may be detected with the inventive assay, including bacteria, fungi, or other microorganisms.

Another embodiment of the invention relates to a method of differentiating Gram-negative bacteria from Gram-positive bacteria comprising the steps of providing a sample potentially containing microorganisms comprising RNA to be analyzed; lysing the microorganisms; extracting the RNA from the microorganisms; incubating the extracted RNA with at least one nucleic acid probe under hybridization conditions so that the at least one nucleic acid probe hybridizes to the extracted RNA to form an RNA-probe complex, wherein the at least one nucleic acid probe is labeled with a detectable label; washing the RNA-probe complex with a wash solution; adding a reagent substrate to the washed RNA-probe complex; and measuring the level of hybridization via the detectable label, wherein detection of the detectable label indicates the presence of a Gram-negative microorganism.

The present invention also relates to a method of detecting the presence of a microorganism in a sample comprising the steps of providing a sample potentially containing microorganisms comprising DNA to be analyzed; lysing the microorganisms; extracting the DNA from the microorganisms; incubating the extracted DNA with at least one nucleic acid probe under hybridization conditions so that the at least one nucleic acid probe hybridizes to the extracted DNA to form a DNA-probe complex, wherein the at least one nucleic acid probe is labeled with a detectable label; washing the DNA-probe complex with a wash solution; adding a reagent substrate to the washed DNA-probe complex; and measuring the level of hybridization via the detectable label, wherein detection of the detectable label indicates the presence of a microorganism.

Another embodiment of the invention relates to a method of differentiating Gram-negative bacteria from Gram-positive bacteria comprising the steps of providing a sample potentially containing microorganisms comprising DNA to be analyzed; lysing the microorganisms; extracting the DNA from the microorganisms; incubating the extracted DNA with at least one nucleic acid probe under hybridization conditions so that the at least one nucleic acid probe hybridizes to the extracted DNA to form a DNA-probe complex, wherein the at least one nucleic acid probe is labeled with a detectable label; washing the DNA-probe complex with a wash solution; adding a reagent substrate to the washed DNA-probe complex; and measuring the level of hybridization via the detectable label, wherein detection of the detectable label indicates the presence of a Gram-negative microorganism.

The assay of the invention may employ at least one fluorosurfactant. The novel use of fluorosurfactants, such as Zonyl® FSA, reduces non-specific binding and foaming of the samples. In addition, novel and beneficial features of the present invention include: the probes used in this assay and their combinations (single probes, dual probes, and triple probes may be used); the relatively short hybridization time; the lack of washes between hybridization and capture steps; the use of a wide range, of hybridization and capture temperatures that show similar results (i.e., about 20° C.-about 42° C., or even up to about 55° C.); and the use of non-stringent washes.

At least one fluorosurfactant employed by the invention may be selected from a group consisting of anionic fluorosurfactants, cationic fluorosurfactants, amphoteric fluorosurfactants, nonionic fluorosurfactants, zwitterionic fluorosurfactants, and mixtures thereof. The at least one fluorosurfactant may be lithium carboxylate salt of 3-[2 (perfluoroalkyl)ethylthio]propionic acid; ammonium bis[2-(perfluoroalkyl)ethyl]phosphate; a perfluoro alcohol with a chain length of less than 4; an ammonium fluoroaliphatic phosphate ester; and mixtures thereof.

In the methods of the invention, at least one nucleotide sequence of interest may be extracted from the sample prior to incubating, if necessary. The extraction can be carried out by incubating the sample with a lysis/ex traction buffer.

The methods of the invention may employ one, two, three, or more nucleic acid probes. The probe may be a single probe that functions as both a capture probe and a signal probe; may be two probes, one of which is a capture probe and the other is a signal probe. Three-probe systems are also contemplated, which will include a capture probe, a signal probe, and a bridge probe. The capture probe and signal probe can be labeled with a detectable label, and a reagent substrate added.

One example of a capture probe label is biotin and an example of the signal probe label is alkaline phosphatase. The alkaline phosphatase will react with a reagent substrate selected from the group consisting of adamantyl-1,2-dioxetane phosphate, 5-bromo-4-chloro-3-indolyl phosphate/nitro blue tetrazolium, and para-nitrophenyl phosphate.

The methods of the invention may comprise a solution phase hybridization followed by a capture of the desired nucleic acid, which is then followed by a wash step. The hybridization step can be performed as either a solution hybridization, as a hybridization on a solid support; or as a hybridization system that utilizes both solution hybridization and hybridization on a solid support.

The methods of the invention may comprise immobilizing the complex, washing the complex with a wash buffer so as to remove unbound material, and retaining the complex before measuring the level of hybridization, via the detectable label. Alternatively, the complex can be immobilized, but not washed. The methods of the invention may also comprise adding a reagent substrate after washing, where a washing step is employed.

The methods of the invention are useful for the detection of both DNA and RNA from all types of samples including, but not limited to, microorganisms, bacteria, and fungi, such as yeast and molds. In addition, the methods of the invention can be used to detect targets of interest other than nucleic acids. For example, the methods can be used to detect proteins, peptides, small chemical molecules, carbohydrates, lipopolysaccharides, polysaccharides, and lipids. The methods can also be used to reduce non-specific binding in applications such as immunoassays, microfluidic assays, cell culture and passivation of vessel surfaces.

Other systems, methods, features, and advantages of the present invention will be or will become apparent to one with skill in the art upon examination of the following figures and detailed description. It is intended that all such additional systems, methods, features, and advantages be included within this description, be within the scope of the invention, and be protected by the claims.

### BRIEF DESCRIPTION OF THE DRAWINGS

The accompanying drawings, which are incorporated in and constitute a part of this specification, illustrate an implementation of the present invention, and together with the description, serve to explain the advantages and principles of the invention. In the drawings:

FIG. 1 depicts a diagram of the secondary structure of the small subunit ribosomal RNA (rRNA) of E. coli.

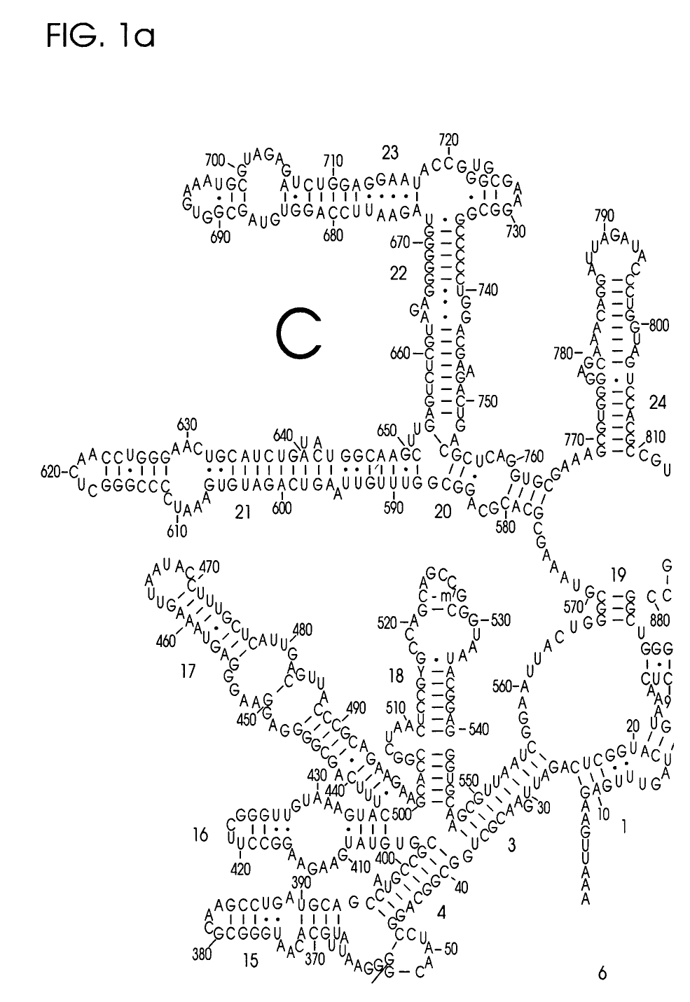

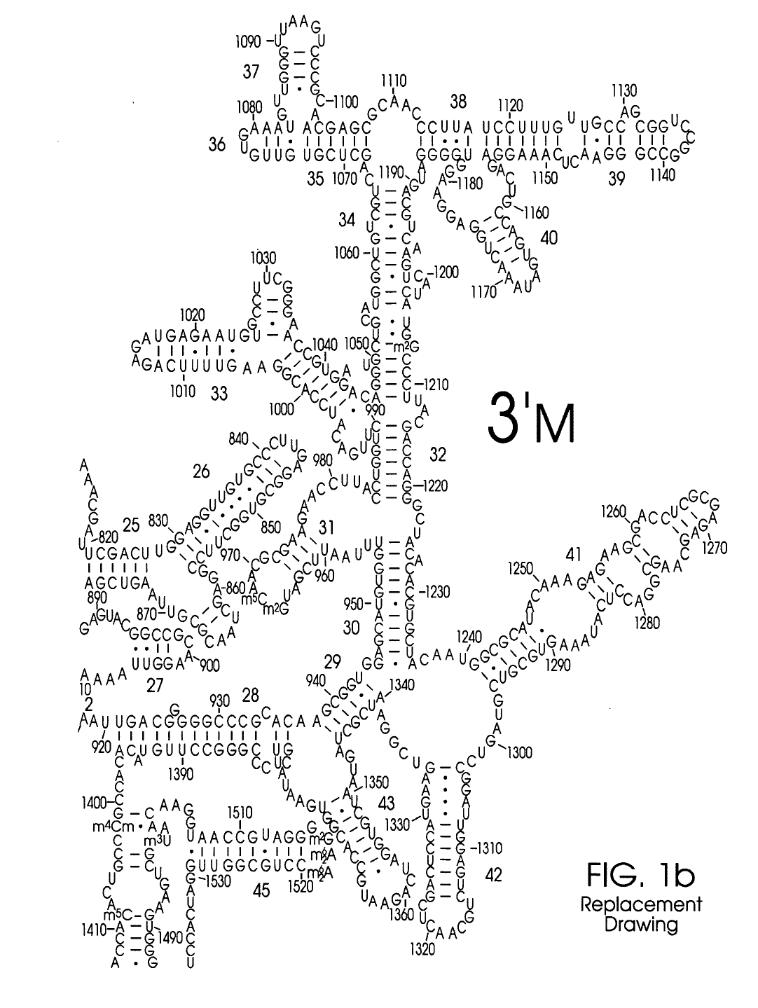

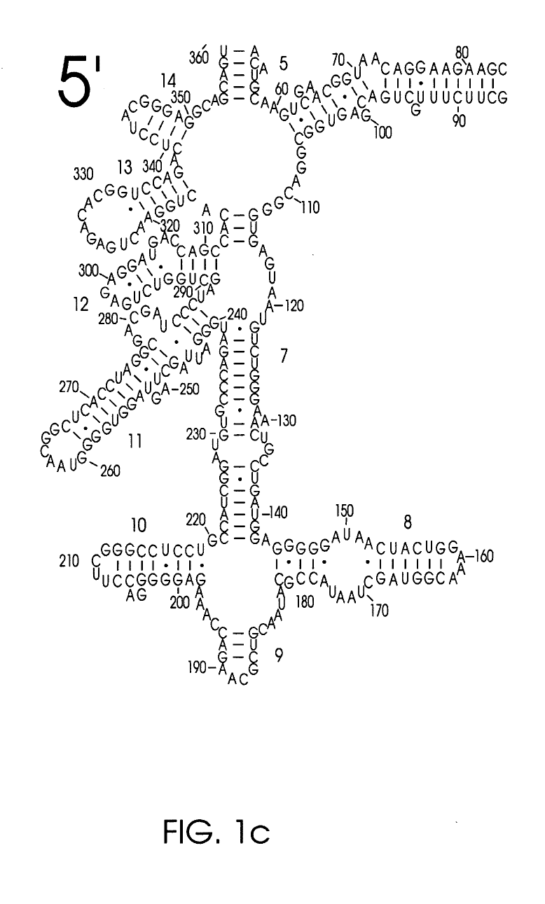

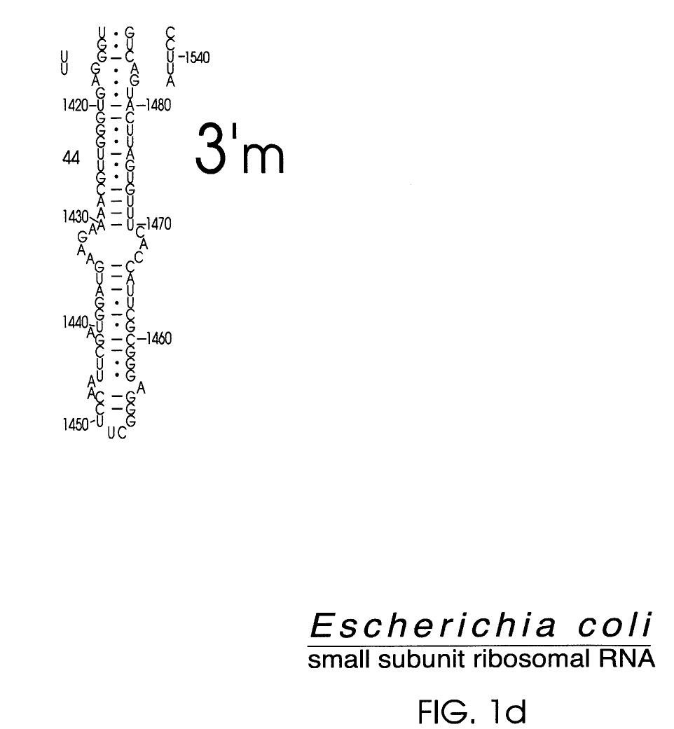

### DETAILED DESCRIPTION OF THE INVENTION

### Definitions

As used herein, the following terms have the given meanings unless expressly stated to the contrary.

A “nucleotide” is a subunit of a nucleic acid consisting of a phosphate group, a 5-carbon sugar, and a nitrogenous base. The 5-carbon sugar found in RNA is ribose. In DNA, the 5-carbon sugar is 2-deoxyribose. For a 5′-nucleotide, the sugar contains a hydroxyl group (—OH) at the 5′-carbon-5. The term also includes analogs of such subunits, and particularly includes analogs having a methoxy group at the 2′ position of the ribose (—OMe). As used herein, methoxy oligonucleotides containing “T” residues have a methoxy group at the 2′ position of the ribose moiety, and a uracil at the base position of the nucleotide.

An “oligonucleotide” is a nucleotide polymer having two or more nucleotide subunits covalently joined together. Oligonucleotides are generally about 10 to about 100 nucleotides in length. The sugar groups of the nucleotide subunits may be ribose, deoxyribose, or modified derivatives thereof, such as OMe. The nucleotide subunits may by joined by linkages such as phosphodiester linkages, modified linkages, or by non-nucleotide moieties that do not prevent hybridization of the oligonucleotide to its complementary target nucleotide sequence. Modified linkages include those in which a standard phosphodiester linkage is replaced with a different linkage, such as a phosphorothioate linkage, a methylphosphonate linkage, or a neutral peptide linkage. Nitrogenous base analogs also may be components of oligonucleotides in accordance with the invention.

A “target nucleic acid sequence,” “target nucleotide sequence” or “target sequence” is a specific deoxyribonucleotide or ribonucleotide sequence that may be hybridized by an oligonucleotide.

A “nucleotide probe” is a nucleotide having a nucleotide sequence sufficiently complementary to its target nucleic acid sequence to be able to form a detectable hybrid probe:target duplex under high stringency hybridization conditions. A nucleotide probe is an isolated chemical species and may include additional nucleotides outside of the target region as long as such nucleotides do not prevent hybridization under high stringency hybridization conditions. Non-complementary sequences, such as promoter sequences, restriction endonuclease recognition sites, or sequences that confer a desired secondary or tertiary structure such as a catalytic active site may be used to facilitate detection using the invented probes. A nucleotide probe optionally may be labeled with a detectable moiety such as a radioisotope, a fluorescent moiety, a chemiluminescent moiety, a chromophoric moiety, an enzyme or a ligand, which may be used to detect, or confirm probe hybridization to its target sequence, or enable a sequence to be identified. In addition, nucleotide probes may contain nucleotide analogs. Nucleotide analog probes include peptide nucleic acid (PNA) probes, probes containing phosphothioates, locked nucleic acid (LNA) probes, and nucleic acid probes containing 2′-O-methyl residues. Nucleotide probes are preferred to be in the size range of 10 to 100 nucleotides in length.

A “signal probe” contains a detectable label. The signal probe is capable of preferentially hybridizing with its target segment within the target nucleic acid from the specimen of interest and is usually comprised of 15-100 nucleotides, frequently 30-70 nucleotides, and preferably 15-29 nucleotides capable of hybridization with the target segment of the target nucleic acid.

A “capture probe” is capable of binding to a solid surface such as nanogold, paramagnetic microparticles, the surface of microliter plate well, the wall of a plastic tube, or a membrane where the solid phase, for example, is coated with a compound. For example, the coating compound may be avidin or streptavidin; the capture probe would then be biotinylated. Additionally, a capture probe is capable of preferentially hybridizing with its target segment within the target nucleic acid from the specimen of interest and is usually comprised of 15-100 nucleotides, though frequently 30-70 nucleotides, but preferably 15-29 nucleotides.

A “bridge probe” is usually unlabeled and capable of preferentially hybridizing with its target segment within the target nucleic acid from the specimen of interest. The bridge probe is usually comprised of 15-100 nucleotides, though frequently 30-70 nucleotides, but preferably 10-29 nucleotides. The bridge probe usually protects the segment of the target rRNA from degradation by the RNase A used in the assay.

The term probe may also encompass a single probe that can be labeled as both a capture probe and a signal probe; a two-probe system with one being the capture probe and the other being a signal probe; or a three-probe system with one being the capture probe, the second being a signal probe, and the third being a bridge probe.

Only one of these probes is required to have absolute discrimination or differential binding to the target nucleic acid. That is, at least one of either the signal or capture probes should have discrimination in hybridization to the selected target segment of the target nucleic acid. The signal probe can be used in a single probe assay. The capture and signal probes can be used in a dual probe assay. The signal capture and bridge probes can be used in a triprobe assay. In practice, the hybridization segments of the signal and capture probes can be substituted for one another, with substantially equal success in discriminating Gram-negative organisms from Gram-positive organisms. When two or more of the probe sets are used in an assay, they will usually hybridize to a contiguous segment of the target nucleic acid, but small gaps may exist between their ends when hybridized to their respective target segments within the target nucleic acid; i.e., usually 10-20 nucleotide sized gaps, more usually 5-10, and preferably none to 3 nucleotides. Sequences and software for probe design are well known in the art and commonly used resources include the nucleic acid repositories GenBank®, and the European Molecular Biology Laboratory (EMBL) and software such as that at the Ribosomal Database Project (RDP) or the ARB Project (ARB).

A “detectable moiety” is a molecule attached to, Or synthesized as part of, a nucleic acid probe. This molecule should be uniquely detectable and will allow the probe to be detected as a result. These detectable moieties are often radioisotopes, fluorescent molecules, chemiluminescent molecules, chromophoric enzymes, haptens, or unique oligonucleotide sequences.

A “hybrid” or “duplex” is a complex formed between two single-stranded nucleic acid sequences by Watson-Crick base pairings or non-canonical base pairings between the complementary bases.

“Hybridization” is the process by which two complementary strands of nucleic acid combine to form a double-stranded structure (“hybrid” or “duplex”).

“Complementarity” is a property conferred by the base sequence of a single strand of DNA or RNA which may form a hybrid or double-stranded DNA:DNA, RNA:RNA, or DNA:RNA through hydrogen bonding between Watson-Crick base pairs on the respective strands. Adenine (A) ordinarily complements thymine (T) or uracil (U), while guanine (G) ordinarily complements cytosine (C).

“Mismatch” refers to any pairing, in a hybrid, of two nucleotides which do not form canonical Watson-Crick hydrogen bonds. In addition, for the purposes of the following discussions, a mismatch may include an insertion or deletion in one strand of the hybrid which results in an unpaired nucleotide(s).

The term “stringency” is used to describe the temperature and solvent composition existing during hybridization and the subsequent processing steps. Under high stringency conditions only highly-complementary nucleic acid hybrids will form; hybrids without a sufficient degree of complementarity will not form. Accordingly, the stringency of the assay conditions determines the amount of complementarity needed between two nucleic acid strands forming a hybrid. Stringency conditions are chosen to maximize the difference in stability between the hybrid formed with the target and the non-target nucleic acid. Exemplary stringency conditions are provided below in the working examples.

The term “probe specificity” refers to a characteristic of a probe's ability to distinguish between target and non-target sequences.

“Bacteria” are members of the phylogenetic group eubacteria, which is considered one of the three primary kingdoms.

“Tin” refers to the temperature at which 50% of the probe is converted from the hybridized to the unhybridized form.

The term “extracted RNA” refers to RNA having an A260/A280 ratio of less than 1.8.

The term “extracted DNA” refers to DNA having an A260/A280 ratio of less than 1.8.

The term “total nucleic acids” refers to total nucleic acids having an A260/A280 ratio of less than 1.8; In purified form, each of the three above cases has an A260/A280 ratio of at least 1.8, usually between 1.8 and 2.2.

One skilled in the art will understand that substantially corresponding probes of the invention may vary from the referred to sequence and still hybridize to the same target nucleic acid sequence. This variation from the nucleic acid may be stated in terms of a percentage of identical bases within the sequence or the percentage of perfectly complementary bases between the probe and its target sequence. Probes of the present invention substantially correspond to a nucleic acid sequence if these percentages are from 100% to 80% or from 0 base mismatches in a 10 nucleotide target sequence to 2 bases mismatched in a 10 nucleotide target sequence. In one embodiment, the percentage is from 100% to 85%. In other embodiments, this percentage is from 90% to 100%; in other embodiments, this percentage is from 95% to 100%.

The term “sufficiently complementary” or “substantially complementary” means nucleic acids having a sufficient amount of contiguous complementary nucleotides to form, under high stringency hybridization conditions, a hybrid that is stable for detection.

The term “nucleic acid hybrid” or “probe:target duplex” means a structure that is a double-stranded, hydrogen-bonded structure, preferably 10 to 100 nucleotides in length, more preferably 14 to 50 nucleotides in length. The structure is sufficiently stable to be detected by means such as chemiluminescent, bioluminescent, or fluorescent light detection, autoradiography, electrochemical analysis, or gel electrophoresis. Such hybrids include RNA:RNA, RNA:DNA, or DNA:DNA duplex molecules or their analogs such as PNAs.

The term “preferentially hybridize” means that under suitable stringency hybridization conditions oligonucleotide probes may hybridize their target nucleic acids to form stable probe:target hybrids (thereby indicating the presence of the target nucleic acids) without forming stable probe:non-target hybrids (that would indicate the presence of non-target nucleic acids from other organisms). Thus, the probe hybridizes to target nucleic acid to a sufficiently greater extent than to non-target nucleic acid, which enables one skilled in the art to accurately detect, for example, the presence of bacteria of the Gram-negative type, such as the family Enterobacteriaceae, and distinguish their presence from that of other organisms. Preferential hybridization may be measured using techniques known in the art and described herein. For example, when compared with hybridization to C. albicans (a yeast) nucleic acids, oligonucleotide probes of the invention preferentially hybridize nucleic acids of bacteria in the family Enterobacteriaceae by about 50-7,000 fold. One of ordinary skill in the art would be able to determine the specific stringency conditions, or range thereof, depending upon their particular situation. That is, the stringency can be determined based upon, but not limited by, the assay being performed, the sources of the nucleic acids, the probes being employed, and the like.

An Enterobacteriaceae “target nucleic acid sequence region” refers to a nucleic acid sequence present in nucleic acid or a sequence complementary thereto found in bacteria of the family Enterobacteriaceae, which is not present in the nucleic acids of other species. Nucleic acids having nucleotide sequences complementary to a target sequence may be generated by target amplification.

The phrase “at least one nucleic acid probe” refers to one or more nucleic acid probes. The nucleic acid may be RNA, DNA, or a nucleotide analog such as PNA.

The term “concurrently” is defined as incubation of all assay components at the same time. More specifically, all components are added to the reaction vessel at the same time, so that all of the required components are present throughout the entire reaction.

Hybridization to RNA, in particular rRNA, is preferred to hybridization to DNA, because RNA single-stranded, usually occurs in high copy numbers, and with the exception of certain viruses, is a proxy for living cells. Even though rRNA is a single-stranded molecule, it has extraordinary secondary and tertiary structures, which can make the access of probes to certain segments extremely difficult. In order to successfully use rRNA in microbial detection assays, the secondary and tertiary structure must be disrupted, but only to the extent that hybridization may take place. If rRNA is denatured in solution, it can nonetheless exhibit snap-back tendencies that will cause reformation of the secondary and tertiary structures. The snap-back occurs rapidly due to intramolecular refolding, which is not diffusion limited. In addition, one must ensure that there is not extensive degradation of the rRNA molecule. That said, rRNAs do make good targets because of their potential to permit differentiation of organisms due to the depth and breadth of their sequence information and their presence and abundance in viable cells.

Applicants have surprisingly and unexpectedly discovered assay conditions and protocols, that enable practitioners of the invention to simply and quickly perform detection and/or determination assays of microorganisms. These conditions include the use of reagents such as a fluorosurfactant. Fluorosurfactants, or fluorinated surfactants, are fluorocarbon-based surfactants that are more effective at lowering the surface tension of water than comparable hydrocarbon surfactants. For the purposes of this specification, the fluorosurfactants may have 6 or more fluorines in a fluorocarbon unit, entity, group or segment portion of the molecule as a whole.

Fluorosurfactants include, but are not limited to, Zonyl® surfactants, by DuPont. These compounds are members of a class of fluorosurfactants and monomers. Zonyl® FSA is the lithium carboxylate salt of 3-[2-(perfluoroalkyl)ethylthio]propionic acid and is represented as R_{f}CH₂CH₂SCH₂CH₂COOLi. Zonyl® FSE is another suitable fluorosurfactant that has similar properties to the Zonyl® FSA above, and is known as ammonium bis[2-(perfluoroalkyl)ethyl]phosphate, and represented by the formula (R_{f}CH₂CH₂O)ₓPO(ONH₄)_{y}(OCH₂CH₂OH)_{3-x-y}. In addition, the fluorosurfactant Zonyl® FSP, which is a mixture of (R_{f}CH₂CH₂O)P(O)(ONH₄)₂ and (R_{f}CH₂CH₂O)₂P(O)(ONH₄), is also useful in the assay of the instant invention. The use of Zonyl® detergents significantly reduces the background signal, enabling the practitioner to obtain more clear and less ambiguous results. In addition, foaming of the samples during the assay is also reduced by the use of Zonyl® surfactants. Other useful fluorosurfactant series are the Surflon® series from Seimi Chemical Co., the Atsurf® series from Imperial Chemical Industries, the PolyFox™ series from Omnova Solutions, which are perfluoro alcohols with chain lengths of less than 4, and the Masurf® series from Mason Chemical Company.

In general, fluorosurfactants can be selected from those having alkyl-, aryl-, and alkyl-aryl-containing perfluorinated segments, which can also contain other functional groups. These groups include, but are not limited to, phosphates, carboxylic acid or amines, which have primary, secondary, tertiary, or quaternary groups or functionalities present in them. Additionally, the fluorosurfactants can be selected from the known major classes of fluorosurfactants, such as anionic, cationic, amphoteric, nonionic, and zwitterionic classes. Specific fluorosurfactants that maybe used in the assays of the invention can be, but are not limited to, lithium carboxylate salt of 3-[2 (perfluoroalkyl)ethylthio]propionic acid; ammonium bis[2-(perfluoroalkyl)ethyl]phosphate; a perfluoro alcohol with a chain length of less than 4; an ammonium fluoroaliphatic phosphate ester; and mixtures thereof.

In particular, anionic fluorosurfactants are useful when the reaction species contain analytes and active reagents, which carry net negative charges at the reaction pH, such as the net negative charge exhibited by the phosphate backbones of nucleic acid probes and sample nucleic acids in hybridization reactions. Frequently, hybridization assays may involve the use of protein interactions. For example, when streptavidin/biotin or avidin/biotin interactions are used, the proteins utilized can have positive charges which cause a charge interaction between the proteins and nucleic acids leading to non-specific binding. Use of an anionic fluorosurfactant can reduce such interactions as demonstrated in the examples below.

Importantly, the beneficial effects of utilizing fluorosurfactants also can be applied to protein-based assays, wherein the primary reaction species have net positive charges at the reaction pH. For example, solution phase or immobilization immunoassays can utilize reaction components such as antibodies, instead of nucleic acids. In these cases, use of cationic fluorosurfactants can reduce such non-specific interactions and reduce background signal. In both nucleic acid hybridization assays and protein based assays, both cationic and anionic as well as nonionic, fluorosurfactants can also be employed to reduce background and non-specific binding, especially with surfaces such as polystyrene, polycarbonate, glass, silica, iron oxides, metals (e.g., gold), or membranes (e.g., those composed of cellulose nitrate, PVDF, cellulose acetate, and the like). Those skilled in the art will appreciate desirable physical properties of useful fluorosurfactants used as assay components, such as water solubility at their intended concentration and compatibility with active biological components (e.g., antibodies or enzymes) so that the fluorosurfactants do not inactivate other assay components.

In certain instances, however, it may be desirable for the fluorosurfactant to inactivate some component in the assay, such as proteases or nucleases, as can readily be determined by those skilled in the art. The type and concentration of the fluorosurfactant will depend on pH, salts, and buffering agents present in the assay, as well as additional detergents and other constituents of the assay. The useful range of fluorosurfactant concentration is generally between 1% and 0.0001%. In some instances, such as for the passivation of a membrane or other solid surface, the fluorosurfactant may be dissolved in an organic solvent or in a water/organic solution (e.g., DMSO or ethanol) and those skilled in the art will appreciate the appropriate solvent conditions as desired. Fluorosurfactants can be added at several points in the assay. In one embodiment, the fluorosurfactant can be added prior to the combining of other ingredients (e.g., as or with a blocking agent). In another embodiment, the fluorosurfactant can be added during the incubation or hybridization step. In yet another embodiment, the fluorosurfactant can be added during a wash step.

In addition, the use of a small amount of RNase surprisingly allows better and more complete hybridization between the RNA and the nucleic acid probes. The use of RNase allows for the disruption of the secondary and tertiary structure of the RNA without extensive degradation of the structure, so that hybridization can occur. While most of the rRNA may eventually be degraded, this degradation should not materially affect the test results. Other nucleases may be employed including, but not limited to non-specific nucleases and DNases. Furthermore, Applicants have surprisingly discovered that the inventive assay may be easily performed using a single assay vessel.

The nuclease is one that will not substantially degrade the sequence of interest or the probe in the complex. The components of the assay, including the sample or at least one sequence of interest, at least one probe labeled with a detectable label, and the nuclease are added to a reaction vessel. The timing of the addition of the nuclease in comparison with the other reaction components may occur before or concurrently with the addition of the sequence of interest and the probe. In one embodiment the nuclease is added at the same time as the sequence of interest and the probe.

The timing of the addition of the nuclease to the hybridization reaction mixture is important, because too early an addition of the nuclease prior to probe addition can lead to degradation of the target sequence in a sample undergoing analysis. When the nuclease is added to the sample suspected to contain the target sequence of interest but before the addition of the one or more nucleic acid probes of the assay, the probes can preferably be added immediately to a few minutes later. For example, the selected time period for when the nuclease can be added from about 0 minutes to about 60 minutes, or from about 1 minute to about 30 minutes, or from about 1 minute to about 15 minutes, or from about 6 minutes to about 5 minutes, or about 0 minutes to about 2 minutes. These time ranges reflect the addition of the nuclease after the addition of the probe and can be important when the addition of the nuclease takes place at temperatures at or near the temperature at which the nuclease is active, this temperature can be about 20° C. to about 50° C. for nucleases derived from mezophilic organisms. Some thermophylic nucleases that are only active at temperatures well above room temperature may exhibit little or no activity at room temperature or lower but have appropriate activity at the hybridization temperature and consequently the timing of the probe addition is less critical.

Another aspect of nuclease addition is that the reaction components can be added in any order under conditions where the nuclease is not active. In this instance, the conditions can be subsequently modified to render the nuclease active without compromising the necessary function of the other assay components. For example, when the temperature of the reaction at the time of component addition is about 4° C. where nucleases generally are inactive, all components can be added virtually simultaneously. A subsequent increase in the assay temperature to that suitable for hybridization will cause the nuclease to regain essentially full activity and allow the hybridization reaction to proceed.

Another embodiment which facilitates the conditional inactivation or activation of the nuclease is the addition of a metal ion. A metal ion inhibits any nuclease used in the assay (e.g., calcium or zinc ions). These ions are added to the buffers for the nuclease at an appropriate concentration considering the metal ion and the nuclease, and thereby reversibly inactivate the nuclease. The components for the hybridization portion of the assay include the sample containing a suspected one or more nucleic acid, one or more nucleic acid probes, and a reversibly inactivated nuclease. Following assembly of the hybridization components the nuclease is activated by the addition of a chelator of the metal ion used. For example, if calcium ions as its chloride salt were used to inhibit the nuclease, then EDTA or EGTA can be used to chelate the calcium and substantially abolish its inhibitory effect on the nuclease. Chelation of the calcium will result in the nuclease substantially regaining its full activity, thus facilitating the hybridization reaction between probes and potential targets. These methods of selective and reversible inactivation of the nuclease facilitate the assembly of reaction components. This is especially useful when large numbers of samples need to be assayed (i.e., 96 samples, 384 samples or 1536 samples and the like) as is frequently the case in high throughput situations. Both embodiments using temperature or chelation can be employed individually or jointly or other combinations of selective reversible inhibition of the nuclease of the assay can be used.

The advantage of selective reversible inhibition of the nuclease means that the assay samples can begin the initiation of the hybridization reaction at the same time. Nuclease activation can occur nearly simultaneously in all samples, thereby improving the quantitative or qualitative results of the assays of the invention when compared to a series of individually assembled reactions where the additions may have substantially different incubation times from the first sample to the last sample in such series.

The assay of the invention may comprise a solution phase hybridization followed by a capture of the desired nucleic acid, which is then followed by a wash step. The inclusion of these steps is in contrast to most organism detection assays, where a wash step following both the initial hybridization and the capture is required. However, the hybridization step can be performed as either a solution hybridization, a hybridization on a solid support, or a hybridization system that utilizes both solution hybridization and hybridization on a solid support.

The assay detection can be carried out by adding a reagent substrate to the sample after washing, where a washing step is employed. In one example, if one nucleic acid probe is used, it can be labeled at one end with biotin and at the other with alkaline phosphatase. If two probes are used, the first, or capture probe can be labeled with biotin, and the second, or signal probe, can be labeled with alkaline phosphatase. In a three probe system, the signal and capture probes can be labeled, with a bridge probe generally being unlabeled. If alkaline phosphatase is used as the signal probe, label, the reagent substrate may be selected from the group consisting of an adamantyl-1,2-dioxetane phosphate, 5-bromo-4-chloro-3-indolyl phosphate/nitro blue tetrazolium, and para-nitrophenyl phosphate. In general, when at least two nucleic acid probes are used, the concentrations of the probes are substantially identical.

Target nucleic acids can be isolated or prepared from numerous sources. In one embodiment, the nucleic acids can be isolated from microorganisms, such as bacteria or viruses. In addition, nucleic acids from multi-cellular organisms can also be used as the target nucleic acid. Alternatively, naked nucleic acids can be employed in the present invention.

If the target nucleic acid requires isolation from a cell or virus, there are numerous nucleic acid isolation protocols that can be used. These protocols can involve lysis of the cell or disruption of a viral envelope and coat using a lysis/extraction buffer. The nucleic acids can be further purified by a variety of techniques, including phenol extraction and ethanol precipitation. These are only brief examples of nucleic acid isolation, and other protocols are well known to those in the art. (See, e.g., “Molecular Cloning—A Laboratory Manual.”).

Once the nucleic acids are isolated or otherwise obtained, the sample might need to be subject to desalting. One well-known method involves the use of NAP columns. To desalt the nucleic acid sample, it is applied to the NAP column, and eluted into a series of tubes. Once the sample is passed through the column, it is further purified using ethanol precipitation. One of skill in the art would additionally be aware of other protocols for the desalting of nucleic acid samples.

If aggregates or precipitates are present in the nucleic acid sample, these can be removed using various well-known protocols. For example, the nucleic acid sample can be subject to membrane filtration, centrifugation, or the like. One of skill in the art would be able to determine the most effective method of particulate/aggregate removal, depending upon the sample and source thereof.

The target nucleic acids can be immobilized to a surface such as nitrocellulose or a glass or plastic microscope slide. In one embodiment, the target nucleic acids are contained in immobilized cells such as formalin fixed paraffin embedded tissues or cells or ethanol fixed and permeabalized cells when RNA is the target suitable considerations are given to preserve the integrity of the RNA. Hybridization is conducted under suitable conditions to effect probe-target duplex formation in the presence of a nuclease and the signal is detected by suitable means considering the nature of the detectable label and its requirements for detection. In addition, the probe, the target, heat stable signal and capture probes, and a heat stable nuclease may be combined and subject to brief exposure to temperature sufficient to denature the nucleotide sequences in the reaction mixture. The mixture can then be subsequently cooled to a temperature at which probes and target nucleotides can form stable discriminatory hybridized duplexes and the nuclease is active. The nuclease is preferably thermophylic or thermostable, or in the case of RNase A, the nuclease can refold and become fully active at the hybridization temperature. For RNA targets exposure should be less than 5 minutes so that the RNA is largely or minimally degraded. Suitable temperatures are on the order of between about 85° C. to about 95° C.

While Applicants are aware that the invention has broad applicability to the detection of both DNA and RNA from a number of different sample types, the invention was evaluated using the Small Subunit (SSU) of ribosomal RNA (rRNA) to optimize the features of the invention. In particular, the SSU rRNAs from bacteria and fungi were utilized in the development of the assay.

In some instances, PCR inhibitors, high levels of nucleases, and other potential assay interferences can pose significant challenges when an assay is scaled-up from the bench of a well-equipped molecular biology laboratory with an experienced staff to real-world manufacturing conditions. In addition, the secondary structure of rRNAs can be challenging because sequence homology can make designing probes difficult to adequately distinguish between species. Furthermore, because of the tenacious secondary and tertiary structures formed by the rRNAs, hybridization with nucleic acid probes can be difficult. Consequently, Applicants have used the SSU rRNA to demonstrate the superiority and novelty of the present invention compared to the present art.

Lysis of microorganisms, if necessary, is carried out using zirconia/silica beads, allowing the RNA to become accessible and subject to hybridization. Lysis can require a buffer system, which may be comprised of 3-(N-morpholino)-propanesulfonic acid (MOPS), ethylenediaminetetraacetic acid (EDTA), SDS, dithiothreitol (DTT), a silicone polymer based antifoam, and a water-dilutable, active silicone (i.e., as designed to control foam in aqueous systems). The composition and concentration of the lysis/extraction buffer can be optimized. For example, the lysis/extraction buffer can be comprised of from about 100 mM to about 300 mM MOPS, about 10 mM EDTA to about 30 mM EDTA, from about 1% SDS to about 3% SDS, from about 5 mM DTT to about 15 mM DTT, from about 0.5% to about 1.5% of a silicone polymer based antifoam and about 0.5% to about 1.5% of a water-disbursable, 30% active silicone emulsion (i.e., as designed to control foam in aqueous systems). Alternatively, the lysis/extraction buffer can be comprised of from about 150 mM to about 250 mM MOPS, about 15 mM EDTA to about 25 mM EDTA, from about 1.5% SDS to about 2.5% SDS, from about 7.5 mM DTT to about 12.5 mM DTT, from about 0.75% to about 1.25% of a silicone polymer based antifoam and about 0.75% to about 1.25% of a water dilutable, 30% active silicone emulsion (i.e., as designed to control foam in aqueous systems). One specific lysis/extraction buffer can be comprised of about 200 mM MOPS, about 20 mM EDTA, about 2% SDS, about 10 mM DTT, about 1% of a silicone polymer based antifoam, and about 1% of a water dilutable, 30% active silicone emulsion (i.e., as designed to control foam in aqueous systems). After lysis, the samples can be filtered through individual syringe filter units to remove cellular debris from the extracted RNA. The samples can be filtered and desalted through the use of such techniques as gel exclusion chromatography, spin columns, and other procedures known in the art.

The extracted RNA is then concurrently incubated under hybridization conditions with nucleic acid probes and a nuclease. Specifically, the instant invention employs at least one probe. In one embodiment, the invention employs a single probe, which may be a signal probe. In another embodiment, the invention employs two probes: a signal probe and a capture probe. In a further embodiment, the invention employs three probes: a capture probe, a bridge probe, and a signal probe. Each of these probes has 16 nucleotides or less of sequence complimentary to the target nucleic acid sequence, and may be comprised of a combination of probes. For example, a combination, or pool, of one or more signal probes may be used. In another example, a combination, or pool, of one or more capture probes may be used. Where such combination probes, or probe sets, are used, more than one sequence of interest can be detected.

When both a capture probe and signal probe are used, and each hybridizes with their respective targets on the same SSU rRNA species, there can be sufficient space of single-stranded RNA between them, so that, if the bridge probe were not in place, one would expect the RNase to cleave this intervening sequence and separate the capture probe and the signal probe into separate RNA-probe complexes, resulting in the subsequent loss of detection in the detection step. However, this loss of detection surprisingly does not occur, even in those instances not employing a bridge probe. In the instant invention, the RNA is not cleaved between the two segments by the action of RNase as one might expect. Therefore, the RNA segments are not separated from one another and the tandem aspect of the capture and signal probes is preserved.

RNase is added during the incubation of the extracted RNA with at least one nucleic acid probe. The RNA will then be disrupted, but only to the point where hybridization will occur. If too much RNase is added, the RNA will be completely degraded. RNase may be added to the incubation in ah amount of between about 10 ng to about 40 ng, or alternatively between about 15 ng to about 25 ng. The amount of RNase may be added to the reaction mixture at about 25 ng.

Useful concentrations of nucleases used in the hybridization assay can be from about 1.0⁻¹² to about 2.0 units, or from about 0.002 to about 0.16 units. For example, for RNase A concentrations or quantities are in the about 0.01 ng to about 1 μg range, or preferably in the about 1 ng to about 80 rig range, especially in the about 4 ng to about 40 ng range for a 150 μl hybridization reaction volume. High quality, or more pure, RNase A has specific activities in the range of 2 units per microgram of pure enzyme based on typical assays for the activity of this enzyme. Those of skill in the art will recognize that such concentrations for the nuclease are dependant upon reaction volumes, target and probe nucleotide concentrations, assay times, and temperatures, as well as the nuclease of combination of nucleases employed in the assay.

In some cases, where discrimination between highly homologous target sequences, such as those differing by a single base requires a substantially higher concentration of the nuclease or nucleases. The range of nuclease concentration to afford such single base discrimination will usually be 2 to 1000 times higher than that described above. In most cases however, the required concentration for highly homologous targets is from 2-100 times. Additionally, nucleases having broader specificity with respect to their strand cleavage motifs may be required to accomplish single base discrimination between target sequences and homologous non-target sequences by the nucleic acid probes used in the assay. For example, when Using an alkaline phosphatase labeled signal probe and a biotinylated capture probe, one optimal RNase A concentration is about 1 ng to about 120 ng, with a reaction temperature of about 20° C. to about 50° C. Those skilled in the art will recognize that different reaction volumes, probe concentrations, and the like will require their own optimization of conditions such conditions being readily determined with reference to this disclosure.

Suitable RNases are RNase A from bovine pancreas, or other equivalent RNases that act on single-stranded RNAs, but not on double-stranded RNAs or RNA-DNA hybrids. RNase A specifically cleaves single-stranded RNA at 3′ phosphate linkages of pyrimidine residues leaving pyrimidine 3′ phosphates and oligonucleotides with terminal pyrimidine 3′ phosphates(1). This enzyme does not require co-factors and divalent cations for activity. Generally, RNase A may be used for the following applications: a) cleaving unhybridized areas of RNA from RNA:DNA hybrids in RNA or DNA mapping; b) removing contaminating RNA from DNA mini-preps; c) preparation of recombinant proteins; d) ribonuclease protection assays; e) plasmid and genomic DNA isolation and f) mapping single-base mutations in DNA or RNA.

Another preferable nuclease is RNase T1 can be isolated from Aspergillus niger, for example, and is an endoribonuclease that specifically degrades single-stranded RNA at G residues. It cleaves the phosphodiester bond between 3′-guanylic residues and the 5′-OH residues of adjacent nucleotides with the formation of corresponding intermediate 2′,3′-cyclic phosphates. The reaction products are 3′-GMP and oligonucleotides with a terminal 3′-GMP. RNase T1 does not require metal ions for activity.

RNase T1 may also be used in a wide variety of applications. RNase T1 may be employed in a) the removal of RNA from DNA preparations; RNA sequencing; b) ribonuclease protection assays; c) conjunction with RNase A; d) the removal of RNA from recombinant protein preparations; and e) determination of the level of RNA transcripts synthesized in vitro from DNA templates containing a “G-less cassette.” Inhibitors of RNase T1 include metal ions and mononucleotides. Guanilyl-2′,5′-guanosine is a specific inhibitor of RNase T1.

A third RNase that may be used in the present invention is RNase I. RNase I may be isolated from Escherichia coli. RNase I degrades single-stranded RNA to nucleoside 3′-monophosphates via 2′, 3′ cyclic monophosphate intermediates by cleaving between all dinucleotide pairs, unlike RNase A, which cleaves only after cytosine and uridine. In addition, the enzyme is completely inactivated by heating at 70° C. for 15 minutes, eliminating the requirement to remove the enzyme prior to many subsequent procedures.

RNase I may be used for the following applications: a) the removal of RNA from DNA preparations; and b) RNase protection assays to detect single-basepair mismatches in RNA:RNA and RNA:DNA hybrids.

Furthermore, one of ordinary skill in the art would understand that the amount of RNase used in an assay may be optimized depending on various criteria. For example, one of ordinary skill in the art would be able to determine the optimal amount of RNase to use in an assay, and to vary this amount when considering certain variables, such as assay volume, the amount of target material present, the assay reaction time, and assay reaction temperature.

S1 nuclease is another enzyme that may be employed in the present invention.

Specifically, S1 nuclease degrades single-stranded DNA and RNA endonucleolytically to yield 5′-phosphoryl-terminated products. Double-Stranded nucleic acids (DNA:DNA, DNA:RNA or RNA:RNA) are resistant to degradation except with extremely high concentrations of enzyme. S1 nuclease may be used to remove single-stranded termini from double-stranded DNA or for selective cleavage of single-stranded DNA and for mapping RNA transcripts or for mapping RNA transcripts.

A nuclease protection assay (NPA) is a laboratory technique used in biochemistry and genetics to identify individual RNA molecules in a heterogeneous RNA sample extracted from cells. This technique can identify one or more RNA molecules of known sequence even at low total concentration. In NPA, the extracted RNA is first mixed with antisense RNA or DNA probes, which are complementary to the sequence or sequences of interest, and these complementary strands are hybridized to form double-stranded RNA (or a DNA-RNA hybrid). The mixture is then exposed to ribonucleases that, specifically cleave only single-stranded RNA, yet exhibit no activity against double-stranded RNA. When the reaction runs to completion, susceptible RNA regions are degraded to very short oligomers or individual nucleotides. The surviving RNA fragments are those that were complementary to the added antisense strand and thus contained the sequence of interest. When the probe is a DNA molecule, S1 nuclease is used; when the probe is RNA, any single-strand-specific ribonuclease can be used. The surviving probe-RNA complement is detected. Nuclease protection assays are used to map introns and 5′ and 3′ ends of transcribed gene regions. Quantitative results can be obtained regarding the amount of the target RNA present in the original cellular extract; if the target is a messenger RNA, this can indicate the level of transcription of the gene in the cell.

The nuclease used in the present invention is capable of degrading the nucleotide sequence of interest in the sample. However, this nuclease may be able to degrade the nucleotide sequence of interest when it is not bound to or present in the complex formed during the reaction. When the complex containing the nucleotide sequence of interest and the nucleic acid probe is formed, the nuclease is unlikely to degrade the sequence of interest or the probe.

The hybridization conditions can be those utilized for solution hybridization, hybridization on a solid support. Alternatively, the hybridization conditions can employ both a solution hybridization component and a solid support hybridization component.

The hybridization reaction can employ a buffer (MOPS, sodium chloride, magnesium chloride, Tween® 20, sodium azide, and an anionic lithium carboxylate fluorosurfactant); probe 1 (SEQ ID NO.: 1; Table 2); and probe 2 (SEQ ID NO.: 2; Table 2). The probes may be present in an amount of about 1 pmole to about 100 pmole per probe, in an amount of from about 2 pmole to about 50 pmole, or in an amount of 5 pmole per probe. The volume of the probe component is dependent upon the final assay volume. The probes are generally used in substantial excess to the target nucleic acid to be analyzed. The probes are usually utilized in equimolar amounts relative to one another. In some circumstances it may be advantageous that the probes are in different mole ratios to one another. For example, in a di-probe hybridization, it may be advantageous to have the capture probe present at a higher concentration than the signal probe, while in other circumstances, an opposite ratio of the probes may be preferable.

The hybridization buffer, the probes, and RNase A are mixed and incubated. The hybridization reaction, as well as the entire assay, may take place at either ambient or elevated temperature. The range of temperatures at which hybridization can take place is about 20° C. to about 55° C. The hybridization reaction may take place at about 31° C., or alternatively at about 42° C.

The hybridization buffer may be comprised of various components at particular concentrations. For example, the hybridization buffer may be comprised of about 100 mM to about 300 mM MOPS, about 1 M to about 3 M sodium chloride, about 0.01% to about 0.1% Tween® 20 (v/v), about 0.005% to about 0.015% sodium azide, and about 0.1% to about 0.3% Zonyl® FSA (anionic lithium carboxylate fluorosurfactant) (v/v). The pH of the hybridization buffer may be between about 6 to about 8 or from about 6.5 to about 7.5 or about 6.9. The hybridization buffer may be comprised of about 150 mM to about 250 mM MOPS, about 1.5 M to about 3.5 M sodium chloride, about 0.02% to about 0.07% Tween® 20 (v/v), about 0.007% to about 0.012% sodium azide, and about 0.15% to about 0.25% Zonyl® FSA (anionic lithium carboxylate fluorosurfactant) (v/v). The hybridization buffer may be comprised of about 200 mM MOPS, about 3 M sodium chloride, about 0.05% Tween® 20 (v/v), about 0.01% sodium azide, and about 0.2% Zonyl® FSA (anionic lithium carboxylate fluorosurfactant) (v/v), with a pH of about 6.9. Other buffers with the above supplements, such as Tris-buffered saline, can be utilized with equal effect, such buffers being well known in the art. The buffer should not contain components that will interfere with detection or inactivate the other assay components, e.g., the exclusion of SDS from hybridization using alkaline phosphatase labeled probe as the SDS would inactivate the enzyme.

Suitable hybridization buffers evaluated for use in the present invention included the following:
TMAC [3 M TMAC, 50 mM MOPS, 0.1% Tween 20, 0.0067% Na azide];
TBS [0.1 M Tris. 0.15 M NaCl, 0.05% Tween 20, 0.01% Na azide];

MOPS/Mannose [50 mM MOPS, 200 mM Mannose, 150 mM NaCl, 0.01% Tween 20];
3 M TMAC, TE (3.3 mM Tris, 0.33 mM EDTA), 0.1% SDS;
0.2 M TEAC, TE (3.3 mM Tris, 0.33 mM EDTA), 0.1% SDS;
SSC (0.15 M NaCl, 0.015 M Na citrate), 0.05% Tween 20;
SSC (0.15 M NaCl, 0.015 M Na citrate), 0.05% Tween 20, 1% PEG high;
SSC (0.15 M NaCl, 0.015 M Na citrate), 0.05% Tween 20, 1% PEG low;
SSC (0.15 M NaCl, 0.015 M Na citrate), 0.05% Tween 20, 10 mM D-Mannose;
0.2 M TEAC;
1 M TEAC;
3 M TMAC;
SSC (0.9 M NaCl, 0.09 M Na citrate), 0.1% SDS; and
1×PGR Buffer (Promega, cat#M8901), 5 mM magnesium chloride

Use of all of the buffers yielded suitable detection and discrimination between different target organisms with probes designed to discriminate between their SSU rRNAs. A preferred embodiment is the hybridization buffer used in example 1. The hybridization buffer, which can be prepared as a 2× concentrate, and then diluted or to a 1× working concentration of the hybridization buffer as used in Example 9.

Another embodiment for the combination of the nuclease with the hybridization components relates to the extent of hybridization between at least one labeled nucleic acid probe and its respective target nucleotide acid sequence, if present in a sample. For example, the nuclease is added after the probe:target hybridization complex, or the percent hybridization, has progressed to about 0% to about 95%, or from about 5% to about 95%, or from about 5% to about 75%, or from about 15% to 65% or from about 15% to about 50%. Alternatively, the percent hybridization can be from about 40% to about 95%. Those skilled in the art will recognize that the extent of hybridization can be utilized when determining the appropriate point for the combination of the nuclease with the other components of the hybridization reaction.

The fluorosurfactant, can be Zonyl® FSA, which reduces both non-specific binding and foaming of the samples during hybridization. Nucleic acid probes are diluted in a dilution buffer prior to hybridization, which may be comprised of Tris, sodium chloride, magnesium chloride, and sodium azide.

Hybridizations can be conducted in solution, phase. However, capture probes may be immobilized to a solid phase rather than captured. Methods for probe immobilization are well known in the art. Suitable solid phase:capture probe embodiments may be nanoparticles in the size range of 10 nanometers to 1 micron with particles in this size range affording reaction and hybridization kinetics highly similar to those obtaining solution phase. Particles suitable for conjugation with capture probes are quantum dots, paramagnetic particles, fluorescently encoded beads, or gold. In one embodiment the capture probe is attached to the walls or wells of a microtiter plate or individual tubes or tube strips. In another embodiment the solid phase is a membrane such as nitrocellulose. In another embodiment the capture probes are immobilized on a flow strip or the like.

Hybridizations can be performed in single tubes especially for dual or tri-probe embodiments with the capture probe immobilized and hybridization and nuclease digestion occurring in the tube followed by washing to remove unbound species and subsequent detection of the signal probe in the target probe set complex. Hybridization can also occur in one tube with subsequent transfer to another tube for capture and detection as demonstrated in the examples. Additionally, another embodiment using single probes is performed by combining a single labeled fluorescent probe, the sample containing or potentially containing the target nucleic acid of the probe and the nuclease followed by denaturation and detection of the hybridized probe:target duplex by fluorescent polarization.

The probes employed in the assay, which can be the signal probes, the capture probes, and the bridge probes, may be diluted to about 0.75 pmoles/μl to about 1.75 pmoles/μl or from about 1 pmole/μl to about 1.5 pmoles/μl. The probes can also be diluted to about 1.25 pmoles/μl with AP (alkaline phosphatase) dilution buffer. The AP dilution buffer may be comprised of about 50 mM to about 150 mM Tris, about 50 mM to about 150 mM sodium chloride, about 1 mM to about 10 mM magnesium chloride, and about 0.001% to about 0.02% sodium azide. The pH of the AP dilution buffer may be about 8 to about 10, or about 8.5 to about 9.5, or about 9.0. The AP dilution buffer may be comprised of about 75 mM to about 125 mM Tris, about 75 mM to about 125 mM sodium chloride, about 2.5 mM to about 7.5 mM magnesium chloride, and about 0.0075% to about 0.015% sodium azide. AP dilution buffer may also be comprised of about 100 mM Tris, about 100 mM sodium chloride, about 5 mM magnesium chloride, and about 0.01% sodium azide and have a pH of about 9.0.

The probes used in the assays of the invention should be resistant or inert to the nuclease or combination of nucleases employed in the assay. The probes can be of DNA composition when RNases are employed in the assay, or RNA when S1 nucleases are employed in the assay. Those skilled in the art would know that the backbone of the nucleotide sequence probes can be modified to be resistant to nucleases in which case the probes can be composed of RNA even when RNase or is used in the assay or they can be comprised of modified nucleotides such as 2′ O-methyl ribonucleotides and the like. Likewise, for either probes directed to DNA or RNA targets, the nucleotide linkages of the backbone can be comprised of phosphothioates, peptide linkages, (e.g., PNAs), or morpholino functionalities. The length of the nucleotide sequences can be from 10-100 nucleotides, 12-30 nucleotides, or 12-25 nucleotides.

Those skilled in the art can appreciate and understand that length and nucleotide composition affect hybridization temperature. Composition and length also affect the specificity of hybridization with the selected target nucleotide sequence and the potential for cross reactivity with non-target nucleotide sequences. This is especially true for homologous target sequences from similar organisms, homologous genes, when the assay is designed to detect small segments of variation (e.g., SNPs in the p53 gene in cancer), or to discriminate from small or large unit ribosomal RNAs of different organisms. In these cases, the probes should be short—usually of a length of 12 to 18 nucleotides—in order to maximize the hybridization of the probe with its target sequence, and to minimize any cross-hybridization with non-target sequences.

In another embodiment, a single probe capable of hybridizing with its target sequence is labeled with a detectable label, such as radioactive P³² or P³³, or a fluorescent label, such as tetramethylrhodamine or CY3. In one embodiment the probe is a single probe with a detectable label. In another embodiment a plurality of individual probes each with an attached individually detectable and individually distinguishable labels are employed to enable the detection of multiple target sequences that may be present in a sample. For example, two probes may be employed to detect the presence of a SNP in a gene where one probe is labeled with CY3 to detect the wild type allele of a target gene and the other probe target to the variant allele. Further, a substantial plurality of probes with individually detectable and distinguishable labels can be prepared limited only by the capability of the method used to discriminate between the labels employed in the multiplex assay using such probes. Importantly when fragment analysis methods are employed to detect the hybridization of the probes with their respective targets the sizes of the formed duplexes can be utilized as an additional means of discrimination between the probe:target duplexes.

As already described, the probes used to detect a target nucleic acid can be composed of a capture probe and a companion signal probe set. In one embodiment a single pair of capture and signal probes are prepared to detect a target nucleotide sequence from other nucleotide sequences in a sample. In another embodiment, a plurality of capture and signal probe pairs can be employed for the detection of multiple target nucleotide sequences within a sample with each signal probe being individually detectable and distinguishable from one another with the considerations as described above for single signal probes.

Similarly, one embodiment for a probe set can be comprised of a capture probe, a bridge probe, and a signal probe set designed to detect a target nucleotide sequence in a sample. As described above for dual probes, another embodiment for the use of triple probes is that a plurality of sets of triple probes designed to detect a plurality of target nucleotide sequences in a sample employed for the detection of multiple target nucleotide sequences within a sample with each signal probe being individually detectable and distinguishable from one another with the considerations as described above for single signal probes.

The probes are preferably used in substantial excess to the anticipated concentration of the target nucleotide sequences in the sample being subjected to analysis usually at 10:1 to 1000:1 ratios of probes to target. Higher probe to target ratios are permitted when signal attributable to background or non-specific biding can be minimized as is understood by those skilled in the art. Additionally, lower ratios of probes to target can be employed; however the time required for hybridization can increase substantially.

When a plurality of probes are employed in the assay, these probes are preferably at similar or nearly identical concentrations to one another, i.e., 1:1. In another embodiment, a plurality of dual or triple probes sets above are used in the assay, where each signal probe can have a common capture probe with the individual detectable and distinguishable signal probes providing the discrimination between different target nucleotide sequences in the sample. In yet another embodiment, the capture probe and the bridge probe may have identical sequences with their companion signal probes, affording the discrimination between different target nucleotide sequences in a sample.

Probes may be provided in a solution phase or in lyophilized form. Additionally when the probes are lyophilized, the nuclease may be co-lyophilized with them, so that on reconstitution they are at appropriate concentration for the assay.

Probe tails, or spacers, are known in the art and previously described. In another embodiment, the tails, or spacers, of probes, are composed of zip code or bar code sequences which allows the probe:target complexes to be captured by their respective zip, or bar, code tails to corresponding zip, or bar, code complimentary sequences immobilized to a surface (e.g., an array) or to fluorescently encoded beads (e.g., beads available from Luminex (Austin, Tex.) or PolyAn Gmbh (Berlin, Del.)).

There are a wide variety of nucleic acid probes that may be employed by the inventive assay. These probes may have spacers attached to either the 5′ or 3′ end of the probe, and these spacers may be used to reduce stearic hindrance during the hybridization reactions. Such spacers can be polyethylene glycol spacers, alkyl chains, or series of 7-10 homologous nucleotides, such as a poly-T chain. Polyethylene glycol and alkyl chain spacers may also be used with biotin. The probes are labeled with a detectable moiety, which is selected from the group consisting of a chemiluminescent label, a bioluminescent label, a radioactive label, a fluorescent label, an enzymatic label, and a chromophoric label. These probes may include specific oligonucleotides, including SEQ ID NOs.: 1-4.

A chemiluminescent label may be selected from the group consisting of alkaline phosphatase (ALP), adenosine triphosphate (ATP), adenylate kinase (AK), luminol, and a luciferase/luciferin combination. The chemiluminescent and bioluminescent labels may be precursors which may ultimately be detected by chemiluminescent and bioluminescent reactions. Upon addition of the reagent substrate, the detectable label will exhibit the characteristic display, enabling the detection of specific microorganisms. The detection of this characteristic display may be accomplished by the use of a luminometer, such as the Celsis Advance™ Luminometer.

When chemiluminescent or bioluminescent labeled probes are employed, the reagent substrate is selected from the group consisting of a luciferase/luciferin/adendosine diphosphate (ADP) combination, a luciferase/luciferin combination, and ATP. In addition, alkaline phosphatase and phosphorylated latent chemiluminescent substrates—including 1,2-dioxetane formulations, such as Lumi-Phos 530, Lumi-Phos 480 and Lumi-Phos Plus offered by Lumigen, Inc. or similar substrates offered by Roche (Indianapolis, Ind.)—and horseradish peroxidase or luminol can be used. Adamantyl-1,2-dioxetane phosphates, or equivalents thereof, are direct substrates for alkaline phosphatase and are sold under tradenames such as Attoglow™ AP Substrate 450LB (Michigan Diagnostics, Royal Oaks, Mich. USA). One advantage of using alkaline phosphatase as the signal-generating enzyme is that alteration in substrates from those visible to the naked eye, such as BCIP/NBT (5-bromo-4-chloro-3-indolyl phosphate/nitro blue tetrazolium) or para-nitrophenyl phosphate, which have a modest sensitivity to chemiluminescent substrates, enables the selection of a wide range of desirable assay sensitivities.

In another embodiment, the buffers and individual components of the assay may contain dyes or other colorants which can serve as visual indications of additions and combinations of reagents to assure their proper assembly, mixing, and pH. Examples of suitable colorants include the seven United States Federal Drug Administration approved FD&C colorants (i.e., FD&C Blue No. 1-Brilliant Blue FCF, E133 (Blue shade), FD&C Blue No. 2-Indigotine, E132 (Dark Blue shade), FD&C Green No. 3-Fast Green FCF, E143 (Bluish green shade), FD&C Red No. 40-Allura Red AC, E129 (Red shade), FD&C Red No. 3-Erythrosine, E127 (Pink shade), FD&C Yellow No. 5-Tartrazine, E102 (Yellow shade), and FD&C Yellow No. 6-Sunset Yellow FCF, E110 (Orange shade)). Suitable concentrations for use as visual indicators often depend on the desired tinting strength and suitability for visualization usually in the 0.001%-0.1% by weight of the dye. Various combinations of the dyes result in a wide variety of colors spanning most of the visual spectrum and provide colors which can be readily distinguished from one another when added to individual reagents and their subsequent combinations resulting from reagent combinations providing different distinguishable colors upon combing the reagents. These color changes provide visual indication of both the proper addition of components and the appropriateness of the sequence of such combinations where a series of reagent combinations are to be performed.

A kit for the detection and determination of nucleic acid sequences of interest and/or microorganisms may be prepared and comprised of at least one nucleotide probe labeled with a detectable label, a fluorosurfactant, and a sequence of interest-degrading nuclease. Alternative embodiments may include a reagent substrate, a lysis/extraction buffer, a hybridization buffer, and/or a wash buffer. The kit may further contain zirconia/silica beads useful in the lysis of microorganisms. If paramagnetic beads are to be used in an assay as described, such beads may be included with the kit. Such paramagnetic beads may be labeled with a probe that may bind to the RNA, with detection being carried out using an additional probe or probes, which also bind to the RNA. Use of liposome-encapsulated luminescent reagents may also be employed, enabling an even greater level of amplification of the detectable signal.

The kit may be further comprised of columns for the clean-up of extracted nucleotide sequences of interest, as well as assay tubes. The assay tubes may be coated with a compound useful in the detection assay, e.g., streptavidin.

The detection assay of the instant invention may be used to detect microorganisms such as bacteria, fungi, or other microorganisms in many different types of samples. In addition, the detection assay of the instant invention may be used to detect nucleic acid sequences of interest in samples. These samples may include food, environmental samples, clinical samples, water, beverages, liquid soaps, sunscreens, cosmetics, toothpaste, fabric softeners, detergents, toners, and personal care products (PCPs). The assay can also distinguish between Gram-negative and Gram-positive bacteria. Should probes specific to Gram-negative bacteria be employed, only this type of bacteria will show a positive result; consequently, the majority of Gram-positive bacteria will not be detected.

The products of the detection assay may also be employed in additional assays such as PCR or RT-PCR. These assays can be used in order to amplify the nucleic acid sequences. This may, in turn, lead to easier and more reproducible detection. Use of PCR or RT-PCR will enable quantitation of the products, as well as increasing the sensitivity of the assays.

The invention further relates to detection assays wherein target of interest include proteins, peptides, small chemical molecules, carbohydrates, lipopolysaccharides, polysaccharides, and lipids. The appropriate probes can be labeled with detectable labels. For example, an antigen can be labeled with radiolabels, chemiluminescent labels, bioluminescent labels, fluorescent labels, or electrochemical labels, and will allow the detection of a corresponding antibody.

In addition, the fluorosurfactants can be employed in many applications to reduce non-specific or unwanted, binding. If at least one fluorosurfactant is added to a buffer or other fluid, as described above, and the application is performed, non-specific binding will be reduced. In addition, the fluorosurfactants will prevent many materials from sticking or binding to a surface or each other. For example, the present invention may relate to a method of reducing non-specific binding of cells, subcellular organelles, biomolecules or chemical molecules comprising: adding at least one fluorosurfactant to a buffer; and contacting the cells, subcellular organelles, biomolecules or chemical molecules with the buffer; wherein the presence of the at least one fluorosurfactant results in the reduction of non-specific binding of the cells, subcellular organelles, biomolecules or chemical molecules to a surface or to each other. This includes, but is not limited to, cells, proteins, peptides, nucleic acids, small chemical molecules, carbohydrates, lipopolysaccharides, polysaccharides, and lipids. This can be a very beneficial effect and result in easier and more reproducible results.

When determining which agents or substances can decrease or substantially reduce non-specific binding, certain desirable properties include, but are not limited to:
Inhibition of non-specific binding (NSB) of assay components to the surface including non-specific hydrophobic, ionic, and covalent binding;
Inhibition of non-specific interactions between assay components, sample components, and surfaces of reaction vessels or container or other surfaces such as membranes;
Lack of cross-reactivity with assay components, especially antibodies and Protein A or G or avidin or streptavidin, nucleic acids, glycosyl groups, fatty acids, lipids, and carbohydrates;
Minimization of the effects of protein denaturation that can occur with phase transitions associated with immobilization and/or drying;
Stabilization of proteins or other molecules when used for diluting reagents that are generally stored refrigerated or frozen;
Low or no contaminating enzyme activity (i.e., peroxidase, alkaline phosphatase);
Inhibition of the enzyme activity in the assay, if present;
No disruption of any immobilized assay components or the detection of a specific protein or biomolecule of interest;
Free of infectious agents; and
Reproducible performance.

The fluorosurfactants described herein possess these properties as exemplified by their description and demonstrated use in the embodiments and examples of the disclosure.

The present invention is described in further detail in the following non-limiting examples. The variety of options falling within the scope of the invention will be readily determinable by those skilled in the art upon consideration of the general methods and kits described above and exemplified below.

### EXAMPLES

### Example 1

### Propagation and Preparation of Microorganism Stock Cultures

The lysis protocol used to obtain nucleic acids suitable for downstream assays was tested using known organisms obtained from the American Type Culture Collection (ATCC®, Manassas, Va. USA).

TABLE 1
Microorganisms ATCC ® Numbers and Gram Stain Status

| Organism Name and | |
|---|---|
| Gram Stain Status = G+ or G− or NA | |
| (If Not Applicable) | ATCC ® Number |
| Escherichia coli | 8739 |
| G− | |
| Bacillus subtilis subsp. Spizizenii | 6663 |
| G+ | |
| Burkholderia cepacia | 25416 |
| G− | |
| Pseudomonas aeruginosa | 9027 |
| G− | |
| Staphylococcus epidermidis | 12228 |
| G+ | |
| Candida albicans | 10231 |
| NA | |
| Saccharomyces cerevisiae NRRL Y-567 | 9763 |
| NA | |
| Aspergillus niger | 16404 |
| WLRI 034(120) | |
| NA | |
| Enterococcus faecium | 35667 |
| G+ | |
| Enterococcus gallinarum | 700425 |
| G+ | |
| Kocuria rhizophila | 9341 |
| G+ | |
| Pseudomonas putida | 49128 |
| G− | |
| Pseudomonas fluorescens | 13525 |
| G− | |
| Ralstonia pickettii AmMS 15S | 49129 |
| G− | |
| Stenotrophomonas maltophilia | 13637 |
| G− | |
| Bacillus cereus | 11778 |
| G+ | |

All organisms with the exception of Aspergillus niger ATCC® 16404 were propagated from the source culture by following the recommendations accompanying the organism stocks from the supplier. After propagation, 30% glycerol stocks of each organism were prepared by adding 1.875 ml of 80% glycerol to 5 ml of confluent microbial growth in appropriate media. The glycerol stocks of each organism were then apportioned into 500 μl aliquots in sterile polypropylene gasketed screw cap 2 ml tubes to provide a set of strain specific archival glycerol stocks. The strain specific archival glycerol stocks were stored at −80° C. until needed for use.

To prepare working glycerol stocks for nucleic acid isolation of each organism, with the exception of Aspergillus niger ATCC® 16404, one tube of strain specific archival glycerol stock was added to 100 ml of Difco™ Letheen Broth [containing lecithin and polysorbate 80 (Tween® 80) (Becton, Dickinson and Company, Sparks, Md. USA)] in a sterile 150 ml Sterile Universal Container (such as part number 1280200, Celsis, Chicago, Ill. or Greiner Bio-One, Monroe, N.C. A) for each organism. The above containers were inoculated with organisms and incubated with the caps tightly closed at 31° C. at 200 rpm on an incubator shaker, such as the C25 Incubator Shaker (New Brunswick Scientific, Edison, N.J. USA) for 24 hours. Working glycerol stocks were prepared using 37.5 ml of 80% glycerol and 100 ml of confluent microbial growth and then apportioned into 1400 μl aliquots in sterile polypropylene gasketed screw cap 2 ml tubes. The working glycerol stocks were stored at −20° C. until needed for use.

Because of its growth characteristics, Aspergillus niger ATCC® 16404 was propagated as follows to provide strain specific archival and working glycerol stocks. Thirty-percent glycerol stocks of Aspergillus niger ATCC® 16404 were prepared by adding 6 ml sterile deionized (DI) water to the ATCC® stock of spores, and then transferred to a 15 ml conical tissue culture tube (Fisher Scientific, Pittsburgh, Pa.) with the cap tightly closed and incubated overnight at room temperature without shaking. Following overnight incubation, 2.25 ml of 80% glycerol was added to provide a 30% glycerol solution of the incubated spores. The 30% glycerol solution of the incubated spores was apportioned into 50 μl aliquots under ultraviolet (UV) light treated 0.5 ml flat cap tubes and a portion of the aliquots to provide working glycerol stock tubes were stored at −20° C. until used, while the remaining aliquoted tubes were used as strain specific archival glycerol stocks and stored at −80° C. until used.

### Species Specific Cultures for Nucleic Acid Isolation

One tube of working glycerol stock for each organism in Table 1 was thawed and transferred into individual sterile 150 ml containers, where each contained 100 ml Letheen Broth. Organisms were grown at 31° C. at 200 rpm on an incubator shaker, such as the C25 Incubator Shaker® for 24 hours. For each organism, the magnitude of growth was determined by analysis on a luminometer, such as the Celsis Advance™ Luminometer, using a luminescence assay for ATP, like the Celsis Rapiscreen™ Reagent (Celsis, Chicago, Ill. USA) following the manufacturer's recommended protocols. Each culture of organisms listed in Table 1 yielded a positive Rapiscreen™ result indicating adequate growth for nucleic acid isolation.

### Lysis of Overnight Cultures

For each organism, 500 μl of overnight growth was added to an individual lysis tube. Each lysis tube (1.5 ml conical screw cap) contained 500 μl lysis/extraction buffer and 1 gram of 0.5 mm zirconia/silica beads. The lysis/extraction buffer was composed of 200 mM 3-(N-morpholino)-propanesulfonic acid (MQPS), 20 mM ethylenediaminetetraacetic acid (EDTA), 2% SDS, 10 mM dithiothreitol (DTT), 1% of a silicone polymer based antifoam (such as Antifoam A (Sigma Aldrich)), 1% of a water dilutable, 30% active silicone emulsion (designed to control foam in aqueous systems) (such as Antifoam Y-30 (Sigma Aldrich) and 100 μM aurintricarboxylic acid (or its salts, such as the ammonium salt), sold as Alumion™ (Sigma Aldrich).

The lysis tubes containing the 500 μl of overnight growth were placed on a pulsing vortex mixer, such as the Deluxe PulseVortex™ Mixer (Fisher Scientific) and vortexed on pulse setting, 3000×rpm for 30 seconds and rest for 10 seconds programmed to repeat this process over the course of 10 minutes.

### Filtration and Column Desalting

After vortexing, each individually lysed sample was poured into its own syringe filter unit consisting of a 0.8/0.2 μm a syringe filter, such as the Acrodisc® Syringe Filter (Pall Corporation, Ann Arbor, Mich. USA), attached to a 3 ml Syringe with locking tip, such as the Luer-Lok™ Tip (Becton, Dickinson and Company, Sparks, Md. USA) and filtered into a Celsis Advance™ Cuvette (12×75 mm polystyrene). A total volume of approximately 700-800 μl of filtrate was collected for each sample in a cuvette. 500 μl of filtrate for each sample was loaded onto a cross-linked dextran gel bead DNA grade column, such as an illustra Nap™-5 column (GE Healthcare, Piscataway, N.J. USA), pre-equilibrated with 1 mM Sodium Citrate pH 6.4. Upon loading the filtrate onto the column, the 500 μl of buffer eluted from the column was discharged as waste. 1 ml of 1 mM Sodium Citrate pH 6.4 was used to elute filtrate from the column. The 1 ml volume of eluate containing the nucleic acids was collected in a Celsis Advance™ Cuvette.

### Gel Electrophoresis

Gel electrophoresis was used to confirm the presence of rRNA in the collected 1 ml column eluate for each sample from above. For each sample, 500 μl of eluate was concentrated by centrifugation using a centrifugal filter unit, such as the Microcon® 30 (Millipore Corporation, Bedford, Mass. USA) at 14,000×g for 25 minutes and backspun at 1,000×g for 1 minute to collect the concentrated sample eluate. Samples were prepared for electrophoresis on a 1.2% agarose gel, such as the FlashGel® RNA Cassette (Lonza, Rockland, Me. USA), using a 2.5 μl aliquot of the concentrated eluate and 2.5 μl Formaldehyde Sample Buffer, which contained formaldehyde, formamide, and tracking dyes in MOPS buffer (Lonza). One microliter of FlashGel® RNA Marker, which contained RNA fragments in sizes from 0.5 to 9 kb (Lonza) and 4 μl Formaldehyde Sample Buffer was used as a size marker. These prepared samples were denatured at 65° C. for 5 minutes and then loaded into individual wells on a FlashGel® RNA Cassette and electrophoresed at 225 volts for 8 minutes. The FlashGel® RNA Cassette stained for approximately 20 minutes following the gel manufacture's suggested protocol. The FlashGel® RNA Cassette was placed oh a high performance ultraviolet transilluminator, and the image was captured with an imaging system designed for photographing gels, such as the Kodak Gel Logic 100® camera (Kodak, Rochester, N.Y., USA). The remaining 500 μl of sample was stored at −20° C. until used in the detection assay.

### Results

A positive result was indicated in the gel photographs by the observation and appearance of rRNA bands of appropriate size and relative intensities, as well as the presence of genomic DNA. These results indicated that the methods used for culture, lysis, and nucleic acid isolation yielded adequate amounts of rRNA and genomic DNA for analysis.

### Detection of Microbial Contamination in the Presence of Personal Care Products

In order to evaluate the utility of the above described methods, a set of representative personal care products (PCPs) were artificially contaminated with five commonly found microbial organisms and tested. Artificial contamination was performed using the following organisms: Escherichia coli ATCC® 8739, Bacillus subtilis ATCC® 6633, Burkholderia cepacia ATCC® 25416, Pseudomonas aeruginosa ATCC® 9027, and Staphylococcus epidermidis ATCC® 12228. Cultures representing non-contaminated and model contaminated personal care products were prepared as follows. Each culture was assembled using one tube of freshly thawed working glycerol stock as described above for each of the five organisms. Three cultures of each of the five organisms were prepared as follows in 150 ml Greiner containers by mixing: 100 ml of Letheen Broth only, Letheen Broth with 1% shampoo (Dove® 2 in 1 Moisturizing Shampoo and Conditioner (Unilever, Trumbull, Conn. USA) (w/v) (I gram shampoo and 99 ml Letheen Broth)), and a tryptone-azolectin-Tween® broth, such as Difco™ TAT broth (Becton, Dickinson and Company) with 1% sunscreen (Banana Boat® Sunscreen SPF 8 (Sun Pharmaceuticals Corporation, Defray Beach, Fla. USA) (w/v) (1 gram sunscreen and 1 gram Tween® 80+98 ml TAT broth). The cultures were incubated at 31° C. at 200 rpm on C25 Incubator Shaker for 24 hours. To measure growth, 50 μl, in duplicates, of 24 hour growth were tested on the Celsis Advance™ instrument using RapiScreen™ assay according to manufacturer's protocol.

The nucleic acids were isolated from the cultures using the method previously described. The 1 ml of column eluate that was collected in a Celsis Advance™ Cuvette was split into 2×500 μl aliquots for each culture. One aliquot of the eluate was concentrated for the purpose of electrophoretic confirmation of the presence of rRNA; all cultures indicated the presence of nucleic acids, in particular rRNA. The other aliquot was used in the detection assay described below.

### Detection Assay

The isolated nucleic acids from the model microbial contaminated cultures described above were assayed by the following method. Briefly, the method consisted of hybridization with a set of probes designed to detect Gram-negative organisms with the nucleic acids isolated from a culture, followed by the capture of the complexes formed by the hybridization of probes with the target rRNA to a solid phase. Following capture of the complexes, the unbound reaction components were removed by washing. Complexes bound to the solid phase were detected by suitable detection methods.

### Hybridization and Capture

For each hybridization reaction, 89.5 μl of the hybridization mix described below was placed in thin-walled 200 μl polymerase chain reaction (PCR) 12 tube strips (Fisher Scientific), along with 60.5 μl of each isolated nucleic acid from the model microbial contamination cultures. The hybridization mix was composed of the following components for each hybridization reaction: 75 μl hybridization buffer [200 mM MOPS, 3 M sodium chloride, 0.05% Tween® 20 (v/v), 0.01% sodium azide, 0.2% Zonyl® FSA (anionic lithium carboxylate fluorosurfactant) (v/v), pH 6.9]; 5 μl probe 1 (SEQ ID NO.: 1; Table 2); 5 μl probe 2 (SEQ ID NO.: 2; Table 2); 2 μl probe 3 (SEQ ID NO.: 3; Table 2); and 2.5 μl RNase A-RPA Grade (freshly diluted to 10 ng/μl with sterile DI water). The probes had been diluted to 1.25 pmoles/μl with AP dilution buffer (100 mM Tris, 100 mM sodium chloride, 5 mM magnesium chloride, 0.01% sodium azide, pH 9.0). The remaining 439.5 μl of isolated nucleic acid was placed at −80° C. Two controls were also assayed: the negative control, which consisted of 60.5 μL of water, and the positive control, which consisted of 1 μl of probe 4 (SEQ ID NO.: 4; Table 2) along with 59.5 μl of water. The tubes containing hybridization mix and nucleic acids of controls were placed on a thermocycler, such as the PTC-200 thermocycler (Bib-Rad, Hercules, Calif. USA) at 42° C. for 30 minutes. The tubes were then removed from the thermocycler, and the contents of each tube were transferred to individual wells of a streptavidin-coated polystyrene plate having eight well strips, such as Reacti-Bind™ Streptavidin Coated High Binding Capacity Clear 8-well Strips (Pierce, Rockford, Ill. USA) with a blocking buffer, such as SuperBlock® Blocking Buffer (Pierce) and incubated for 30 minutes at room temperature to allow hybridization and capture to the plate. Each well was then washed with 250 μl of wash buffer [100 mM Tris, 150 mM sodium chloride, 0.05% Tween® 20 (v/v), 0.01% sodium azide, 0.1% Zonyl® FSA (v/v) pH 7.2] and sonicated for 1 minute. The well contents were then discarded with a flicking motion to remove essentially all of the liquid; the washing process was repeated five times with 250 μl of wash buffer and 1 minute sonication for each wash. Sonication was performed by placing the tubes in an improvised floating rack placed in a jewelry cleaner, such as the Model 840 Jewelry Cleaner (Standard Products Corp.; Whitman, Mass., USA), using water for floatation.

TABLE 2
Probes Used in Detection Assay

| Probe | | | | Probe |
|---|---|---|---|---|
| number | Probe Name | Probe Sequence | Probe Buffer | Concentration |
| 1 | Capture Probe | 5′-/5biotin/TTT TTT TTT TTA | 0.1X Tris-EDTA (TE) | 1 pmole/μL |
| | (IDT,Coralville, | TTA CCG CGG CTG CT-3′ | (EMD), | |
| | IA USA) | (SEQ ID NO.: 1) | 2% (v/v) Acetonitrile | |
| | | | (Sigma-Aldrich) | |
| 2 | Bridge Probe | 5′-GGC ACG GAG TTA GC-3′ | 0.1X TE (EMD), | 1 pmole/μL |
| | (IDT) | (SEQ ID NO.: 2) | 2% (v/v) Acetonitrile | |
| | | | (Sigma-Aidrich) | |
| 3 | AP Signal Probe | 5′-CGG TGC TTC TTC TGC | BioVentures AP | 125 pmole/μL |
| | (BioVentures, Inc.) | GTT TTT TTT TT-3′ | Preservation Buffer | |
| | | (SEQ ID NO.: 3) | | |
| 4 | Gram-Negative | 5′-GTT ACC CGC AGA AGA | 0.1X TE (EMD), | 0.5 pmole/μL |
| | Positive Control | AGC ACC GGC TAA CTC CGT | 2% (v/v) Acetonitrile | |
| | (IDT) | GCC AGC AGC CGC GGT AAT | (Sigma-Aldrich) | |
| | | ACG G-3′ (SEQ ID NO.: 4) | | |

### Substrate Incubation

The washed wells were then treated with substrate as follows: following the final wash, 200 μl of disodium 3-(4-methoxyspiro{1,2-dioxetane-3,2′-(5′-chloro)tricyclo[3.3.1.13.7]decan}-4-yl)phenyl phosphate (CSPD® substrate) (Roche Diagnostics, Indianapolis, Ind. USA) was added to each well. The substrate was incubated in each well for 20 minutes at room temperature while protected from light with aluminum foil. At the completion of the substrate incubation, the 200 μl of the incubated substrate was transferred into a Celsis Advance™ Cuvette. The cuvette was placed in the Celsis Advance™ Luminometer instrument. The instrument was operated with the following parameters: no injectors were used, 10 second read, zero cal cutoff, and 0.6 cal factor for relative light units (RLU). RLU corresponds to arbitrary relative light units and is a measure of emitted light detected by the instrument.

### Detection Assay Results

TABLE 3
Results of Detection Assay.

| Sample ID | RLU1 | RLU2 | AVG RLU | Ratio | Gram Status |
|---|---|---|---|---|---|
| Roche CSPD ® only | 150 | 133 | 142 | — | N/A |
| Negative control | 468 | 433 | 451 | 1.0 | N/A |
| Positive control | 79183 | 78559 | 78871 | 174.9 | N/A |
| E. coli Letheen | 15136 | 17082 | 16109 | 35.7 | negative |
| B. cepacia Letheen | 6887 | 7558 | 7223 | 16.0 | negative |
| P. aeruginosa Letheen | 37787 | 39697 | 38742 | 85.9 | negative |
| B. subtilis Letheen | 434 | 495 | 465 | 1.0 | positive |
| S. epidermidis Letheen | 460 | 879 | 670 | 1.5 | positive |
| E. coli Letheen/shampoo | 16882 | 15489 | 16186 | 35.9 | negative |
| B. cepacia Letheen/shampoo | 5404 | 4191 | 4798 | 10.6 | negative |
| P. aeruginosa Letheen/shampoo | 27604 | 26470 | 27037 | 59.9 | negative |
| B. subtilis Letheen/shampoo | 341 | 370 | 356 | 0.8 | positive |
| S. epidermidis Letheen/shampoo | 434 | 516 | 475 | 1.1 | positive |
| E. coli TAT/sunscreen | 12119 | 9452 | 10786 | 23.9 | negative |
| B. cepacia TAT/sunscreen | 4686 | 4432 | 4559 | 10.1 | negative |
| P. aeruginosa TAT/sunscreen | 40992 | 51046 | 46019 | 102.0 | negative |
| B. subtilis TAT/sunscreen | 430 | 398 | 414 | 0.9 | positive |
| S. epidermidis TAT/sunscreen | 422 | 500 | 461 | 1.0 | positive |

The cutoff for a negative result was set as two times the average negative control RLU (RLU 902 cutoff). All of the model microbial contaminated cultures that were inoculated with Gram-negative organisms had an average RLU that was more than two times the negative control average RLU, giving a positive result. All of the model microbial contaminated cultures inoculated with Gram-positive organisms had an average RLU that was less than two times the negative control average RLU, giving a negative result. This detection assay provides discrimination of Gram-negative organisms over Gram-positive organisms grown in broth only as well as broth plus representative personal care products.

Surprisingly, the presence of RNase provided adequate access for hybridization to occur between the probes and the non-denatured ribosomal RNAs examined without the requirement to denature the ribosomal RNAs examined. That is, neither the use of chaotropic reagents or heat sufficient to denature the ribosomal RNAs was required to permit hybridization of the probes with the ribosomal RNAs. Without the addition of RNase A, signals essentially identical to the negative control were obtained for ribosomal RNAs examined under essentially identical conditions as described above. Further and unexpectedly, ribosomal RNAs examined in the absence of the bridge probe (sequence 2 (SEQ ID NO.: 2) in Table 2 above) yielded approximately only 20% less signal for Gram-negative organisms with no effect on values obtained for Gram-positive organisms which were near those of the negative control. These same sets of capture and signal probes directed to ribosomal RNAs provided suitable distinction between Gram-negative and Gram-positive organisms in the absence of the bridge probe. In addition, it was observed that mild sonication improved the reproducibility of the signals obtained for both controls and sample ribosomal RNAs, when compared to samples processed in the absence of sonication.

Using essentially the method of Example 1, a larger set of representative personal care products (PCPs) were artificially contaminated with the following five commonly found microbial organisms: Escherichia coli ATCC® 8739, Bacillus subtilis ATCC® 6633, Burkholderia cepacia ATCC® 25416, Pseudomonas aeruginosa ATCC® 9027 and Staphylococcus aureus ATCC® 6538. Cultures representing non-contaminated and model contaminated personal care products were prepared in 150 ml Greiner containers by mixing: 100 ml of Letheen Broth only; 100 ml Letheen Broth with 1 g of the following products:
Body Wash (Dove® Beauty Body Wash (Unilever, Trumbull, Conn. USA)) Sunscreen (Banana Boat® Sunscreen SPF 8 (Sun Pharmaceuticals Corporation, Delray Beach, Fla. USA))
Cough medicine (Vicks 44 Custom Care (Proctor and Gamble, Cincinnati, Ohio USA))
Mascara (L'Oreal Voluminous (L'Oreal USA Inc., New York, N.Y. USA))
Toothpaste (Colgate Cavity Protection (Colgate-Palmolive Company, New York, N.Y. USA))
Fabric Conditioner (Suavitel Field Flowers (Colgate-Palmolive Company, New York, N.Y. USA))
Multi-surface Cleaner (Mr Clean Summer Citrus (Proctor and Gamble, Cincinnati, Ohio USA))
Milk of Magnesia (Philips (Bayer Healthcare, Morristown, N.J. USA))
Oatmeal Bath Treatment (Aveeno (Johnson & Johnson, Skillman, N.J. USA)).

Broth controls and broths with added products were inoculated with each of the 5 species listed above, using a quantitative microbial preparation (Quanticult® (Remel Inc., Lenexa, K R USA)), such that each inoculated sample received less than 100 viable microbial cells. The samples were incubated at 30-32° C. in an Incubator Shaker at 200 rpm for 24 hours.

For each organism, the magnitude of growth was determined by analysis on a luminometer, such as the Celsis Advance™ Luminometer, using a luminescence assay for ATP, like the Celsis Rapiscreen™ Reagent Kit (Celsis, Chicago, Ill. USA), and a luminescence assay for the marker enzyme adenylate kinase (AK), like the Celsis AKuScreen™ Reagent Kit (Celsis, Chicago, Ill. USA), following the manufacturer's recommended protocols. Each inoculated sample yielded positive Rapiscreen™ and AKuScreen™ results, indicating adequate growth for nucleic acid isolation. With one exception, each un-inoculated sample yielded negative results, indicating the absence of any unintended contamination. The exception was the Oatmeal Bath Treatment, which was found to contain viable spores.

Desalting spin columns (BioVentures, Inc.) were prepared for storage buffer removal by twisting off the bottom tab, turning the top screw cap one-quarter turn and then placing each of the columns into a collection tube consisting of a Celsis Advance™ Cuvette (55 mm×12 mm polystyrene tube) and centrifuging each column-tube assembly at 1,000×g for 1 minute. Collection tubes were discarded as waste and each of the Centrifuged spin columns was then transferred to a fresh Celsis Advance™ Cuvette to form a spin column-tube assembly. The cell lysates obtained by pulse vortexing were then desalted using 100 μL of each lysate by transfer to an individual and corresponding prepared spin column-tube assembly with each lysate processed in duplicate. Each assembly with applied lysate was then centrifuged at 1000×g for 2 minutes and the column filtrate was collected in the tube portion of the assembly. Each applied lysate yielded ˜100 μL of filtrate in its respective collection tube.

For each hybridization reaction, 75 μl of the hybridization mix described below was placed in thin-walled 3.5 ml polypropylene tubes (Simport), along with 75 μl of each isolated nucleic acid from the inoculated and un-inoculated samples described above. The hybridization mix was composed of the following components for each hybridization reaction: 75 μl hybridization buffer [200 mM MOPS, 3 M sodium chloride, 0.05% Tween® 20 (v/v), 0.01% sodium azide, 0.2% Zonyl® FSA (anionic lithium carboxylate fluorosurfactant) (v/v), pH 6.9]; 5 μl probe 1 (SEQ ID NO.: 1; Table 2); 5 μl probe-2 (SEQ ID NO.: 2; Table 2); and 2 μl probe 3 (SEQ ID NO.: 3; Table 2); and 2.5 μL RNase A-RPA Grade (freshly diluted to 10 ng/μl with sterile DI water). The probes had been diluted to 1.25 pmoles/μl with AP dilution buffer (100 mM Tris, 100 mM sodium chloride, 5 mM magnesium chloride, 0.01% sodium azide, pH 9.0).

Two controls were also assayed: the negative control, which consisted of 75 μL of the hybridization mix and 75 μL 1 mM Sodium Citrate pH 6.4, and the positive control, which consisted of 75 μL of the hybridization mix, 75 μL 1 mM Sodium Citrate pH 6.4, and 1 μl of probe 4 (SEQ ID NO.: 4; Table 0.2). The tubes containing hybridization mix and nucleic acids or controls were placed in a heating block (Troemner, Thorofare, N.J. USA) at 42° C. for 30 minutes.

The contents of each tube were then transferred to individual streptavidin-coated polystyrene tubes (Microcoat, Bernried, Germany) and incubated for a further 30 minutes at 42° C. to allow capture. Each well was then washed with 1 ml of wash buffer [100 mM Tris, 150 mM sodium chloride, 0.05% Tween® 20 (v/v), 0.01% sodium azide, 0.1% Zonyl® FSA (v/v) pH 7.2], vortexing all tubes. Tube contents were discarded with a flicking motion to remove essentially all of the liquid and the washing process was repeated a 3 more times.

The washed tubes were then treated with substrate as follows: following the final wash, 200 μl of chemiluminescence substrate (SAP1113 (Michigan Diagnostics, Royal Oak, Mich. USA) was added to all tubes, which were immediately placed in the Celsis Advance™ Luminometer instrument. The instrument was operated with the following parameters: no injectors were used, 10 second read, zero cal cutoff, and 0.6 cal factor for relative light units (RLU). RLU corresponds to arbitrary relative light units and is a measure of emitted light detected by the instrument.

As found with the previous example, the model microbial contaminated cultures that were inoculated with Gram-negative organisms had an average RLU that was generally more than two times the negative control average RLU, giving positive results. The model microbial contaminated cultures inoculated with Gram-positive organisms had an average RLU that was generally less than two times the negative control average RLU, giving negative results.

### Example 2

### PCR Validation of the Results in Example 1

To confirm the results achieved in the detection assay of Example 1 (Table 3), polymerase chain reaction (PCR) amplification was performed as a validation method. PCR amplification was performed on each of the isolated nucleic acids from the model microbial contamination cultures of Example 1 using two Gram-negative specific primers. The sequences of the forward and reverse primers used consisted respectively of 5′-CCG CAG AAG AAG CAC CGG C-3′ (SEQ ID NO.: 5) and 5′-TGT RTG AAG AAG GYC T-3′ (SEQ ID NO.: 6) both from IDT. R is defined as a purine (adenine of guanine). Y is defined as a pyrimidine (thymine or cytosine).

Each of the frozen isolated nucleic acids from the model microbial contaminated cultures of Example 1 were removed from −80° C., thawed, and then were used as templates by making 1:100 dilutions in sterile, deionized water. PCR amplification was performed in duplicate on each of the fifteen frozen nucleic acids. The PCR reaction components were assembled in the following manner for 20 μl reactions: 2 μl (10%) 10×PCR buffer (ABI, Foster City, Calif., USA) (2 mM magnesium chloride, 2% dimethylsulfoxide (DMSO); 5 mM dithiothreitol (DTT); 200 μM dNTP); 0.125 μl of Taq DNA polymerase (AmpliTaq Gold® 9 at 5 U/μl (Applied Biosystems, Foster city, Calif., USA)); 0.2 μl (10 pmoles) of reverse primer (SEQ ID NO.: 6) at 100 pmoles/μl in 0.1×Tris-EDTA (TE), 2% acetonitrile, 0.2 μl (10 pmoles) of forward primer (SEQ ID NO.: 5) at 100 pmoles/μl in 0.1×TE 2% acetonitrile; and q.s. to 20 μl with sterile DI water PCR components were combined in a 2.0 ml tube and gently mixed. The PCR mix was dispensed using 20 μl of the PCR mix in each of 30 wells of a 96-well PCR plate (Multiplate®, Bio-Rad, Hercules, Calif., USA). Template nucleic acids were added using 1 μl of each 1:100 template dilution to the appropriate well. A film sealant, such as Microseal® Film (Bio-Rad) was used to seal the PCR plate. PCR was performed on a PTC-200 thermal cycler (Bio-Rad) as follows: denaturation at 95° C. for 12 minutes, then 30 cycles of 95° C. for 30 seconds, 60° C. for 20 seconds, 72° C. for 40 seconds, then a final extension at 72° C. for 6 minutes, and a then 4° C. hold. Upon completion of the program, 2 μl of each reaction was analyzed by gel electrophoresis on a 20 well 4% gel of a high resolution, standard melting point agarose, such as NuSieve® 3:1 Plus Agarose gel (Cambrex, Rockland, Me., USA) at 200 volts for 25 minutes. The agarose gel was placed on a high performance ultraviolet transilluminator and the image was captured with a Kodak Gel Logic 100 camera. Size of PCR products was estimated using DNA size markers, such as BioMarker® MIA (BioVentures, Inc.).

### Results

The Gram-negative organisms, E. coli and P. aeruginosa, were positive for each PCR reaction. The PCR amplification for the Gram-negative organism, B. cepacia, was very weakly positive for Letheen Broth/1% shampoo and TAT Broth/1% sunscreen, but no band was observed in the Letheen Broth only. PCR amplification for the Gram-positive organisms, B. subtilis and S. epidermidis, were negative for each PCR reaction. To verify that each of the Gram-positive organisms were truly negative, an additional 7 cycles of the PCR was performed. The PCR plate containing the remaining 18 μl of each reaction was placed on the PTC-200 thermocycler and the plate was sealed with Microseal® film. PCR amplification was performed for 7 additional cycles, which resulted in 37 PCR cycles total. Upon completion of the program, 2 μl of each PCR reaction was analyzed by gel electrophoresis as described above.

The Gram-negative organisms, E. coli, P. aeruginosa, and B. cepacia, were positive for PCR after 37 cycles, as exhibited by the presence of bright bands on the 4% NuSieve® 3:1 Plus Agarose gel. Each of the Gram-positive organisms, B. subtilis, and S. epidermidis, exhibited no band on the 4% NuSieve® 3:1 Plus Agarose gel.

These results demonstrate that the methods of Example 1 may be used to isolate nucleic acids and to provide genomic DNA suitable for use in PCR. The results also demonstrate that Gram-negative specific primers distinguished between Gram-negative and Gram-positive organisms. Additionally, the DNA isolated from the lysis protocol, as described above, has been demonstrated to be suitable for PCR.

### Example 3

Referring to Example 1, Applicants surprisingly observed that Zonyl® FSA surfactant used in the hybridization and wash buffers surprisingly exhibited superior reduction of non-specific background signal as compared to traditional, non-specific blocking agents experimentally evaluated by Applicants, including bovine serum albumin, and detergents such as Tween® 20, SuperBlock® Blocking Buffer, Denhardt's solution, and various polyethylene glycols. All of these traditional blocking agents were used at concentrations consistent with accepted literature methods and all gave unacceptably high backgrounds as compared to those achieved with the Zonyl® FSA as used in Example 1. Applicants observed significant foaming and interference arising from vortexing and/or shaking when using traditional detergents or surfactants in hybridization protocols. This foaming can interfere with the complete removal of the buffer solutions used for hybridization and/or washing. Also, Applicants surprisingly found that Zonyl® FSA surfactant had some foaming upon shaking which rapidly dissipated, as well as producing little, if any, foaming when strongly vortexed even in the presence of Tween® 20 as set forth in Example 1. Consequently, Zonyl® FSA surfactant was used according to Example 1, because its use reduced non-specific background and facilitated improved removal of buffers by essentially eliminating interference arising from foaming.

### Example 4

### RNase A

An experiment was conducted to determine the optimal amount of RNase A to use during hybridization. The following RNase A-RPA Grade dilutions were made in sterile DI water: 1 ng/μl, 200 ng/μl, 40 ng/μl, 8 ng/μl, and 1.6 ng/μl. A hybridization mix was made with the following components per reaction: 75 μl hybridization buffer [200 mM MOPS, 3 M sodium chloride, 0.05% Tween® 20 (v/v), 0.01% sodium azide, and 0.2% Zonyl® FSA (v/v), pH 6.9]; 10 μl E. coli rRNA at 100 ng/μl; 5 μl probe 1 (SEQ ID NO.: 1); 2 μl of a 1:100 dilution of the AP signal probe [(5′-CGG TGC TTC TTC TGC GTT TTT TTT TT-3′ (SEQ ID NO.: 3), conjugated with alkaline phosphatase at 250 pmoles/μl in AP preservation buffer (3 M sodium chloride, 30 mM Tris, 1 mM magnesium chloride, and 0.1 mM zinc chloride) and diluted in AP dilution buffer (100 mM Tris, 100 mM sodium chloride, 5 mM magnesium chloride, 0.01% sodium azide, pH 9.0, and 0.15 μl of Zonyl® FSA)]; 5 μL probe 2 (SEQ ID NO.: 2); and 51.9 μl sterile DI water to bring the hybridization mix volume up to 149 μl. Two reactions were used for every RNase A dilution for a total of 10 reactions. Hybridization mix was added to 10 wells of Reacti-Bind™ Streptavidin Coated High Binding Capacity Clear 8-well Strips with SuperBloc® Blocking Buffer (149 μl hybridization mix per well). A microliter of each RNase A dilution was added to each duplicate. The strips were placed in a water bath at 31° C. for 30 minutes. Each reaction was then washed with 250 μl of wash buffer [100 mM Tris, 150 mM sodium chloride, 0.05% Tween® 20 (v/v), 0.01% sodium azide, 0.1% Zonyl® FSA (v/v), pH 7.2]. The wash buffer was then discarded with a flicking motion to remove essentially all of the liquid; the washing process was repeated five times using 250 μl of wash buffer for each wash. Attoglow™ AP Substrate-450LB was diluted 1:10 with AP dilution buffer, 200 μl of the diluted substrate was added to each well and incubated for 20 minutes at room temperature protected from light. The 200 μl of substrate was then transferred to Celsis Advance™ cuvettes. The cuvettes were placed in the Celsis Advance™ Luminometer instrument. The instrument was operated with the following parameters: no injectors were used, 10 second read, zero cal cutoff, and 0.6 cal factor for RLU.

The results indicated that between 10-40 ng of RNase A is optimal for the parameters noted above.

### Example 5

### 1 mM Sodium Citrate Equilibrated Illustra™ Nap-5 Column vs. Non-Equilibrated Illustra™ Nap-5 Column

The purification of the lysate, as described in Example 1, had been accomplished with an Illustra™ Nap-column that was pre-equilibrated with 1 mM sodium citrate, pH 6.4. To easily manufacture and assemble a final kit configuration, Applicants prefer to avoid pre-equilibration of the column.

A comparison of pre-equilibrated Illustra™ Nap-5 and non-equilibrated Illustra™ Nap-5 columns was performed. Lysis and filtration were performed as previously indicated in Example 1 with the exception of using both equilibrated and non-equilibrated Illustra™ Nap-5 columns in the filtration process. Samples were analyzed using the detection assay as described in Example 1.

A positive result was considered to be 2 times above the average negative control RLU. Gram-negative organisms, S. maltophilia, R. pickettii and B. cereus, were all positive, while the Gram-positive organism, B. cereus, was negative. While all non-equilibrated sample's RLUs were slightly lower, they were not significantly low enough to warrant the extra modification of equilibrating the columns, Illustra™ Nap-5 or equivalent columns prepared in aqueous suspension with a solution of a broad spectrum biocide, such as 0.15% Kathon™ CG/ICP Biocide, are suitable for nucleic acid cleanup.

### Example 6

### Comparison of Streptavidin Coated Cuvettes and Pierce Streptavidin Coated Wells for Capture of Hybridization Complex

A comparison of Pierce Reacti-Bind™ Streptavidin Coated High Binding Capacity Clear 8-well Strips with SuperBlock® Blocking Buffer versus Streptavidin Coated cuvettes was completed in order to determine if any gains in signal and ratio over background could be achieved.

Fresh nucleic acid isolates of E. coli, B. cepacia, B, subtilis, and S. epidermidis grown in Letheen broth were prepared by following the steps outlined in the sample preparation, lysis, and filtration sections according to Example 1.

A hybridization mix with the following components was assembled for each hybridization reaction: 75 μl hybridization buffer [200 mM MOPS, 1 mM magnesium chloride, 3 M sodium chloride, 0.05% Tween® 20 (v/v), 0.01% sodium azide, 0.2% Zonyl® FSA (v/v), pH 6.9, RNase A —RPA Grade at 25 ng/75 μl]; 5 μl probe 1; and probe 2 mixture (SEQ ID NO.: 1 and SEQ ID NO.: 2, respectively) (probe 1 and probe 2 had been combined at a concentration of 1 pmole each/μl); 2 μl of 1.25 pmol/μl probe 3 (SEQ ID NO.: 3) (Table 2) with AP preservation buffer [3 M sodium chloride, 30 mM Tris, 1 mM magnesium chloride, 0.1 mM zinc chloride, 0.05% sodium azide].

For the hybridization reaction, 82 μl of the hybridization mix was placed in a polypropylene cuvette, along with 68 μl of isolated nucleic acid sample. Two controls were also assayed: the negative control consisted of 68 μl of water, and the positive control consisted of 1 μl of probe 4 (SEQ ID NO.: 4) along with 67 μl of water. The polypropylene cuvettes containing hybridization mix and sample or control were placed on a modular block dry-bath incubator, such as the Isotemp® modular block dry-bath incubator (Fisher Scientific), at 42° C. for 30 minutes. The cuvettes were then removed from the dry-bath incubator and the samples were transferred to either a Pierce Reacti-Bind™ Streptavidin Coated High Binding Capacity Clear 8-well Strips with SuperBlock® Blocking Buffer or streptavidin coated cuvette. The different cuvette and well sizes require each to be treated in a different manor as described below.

### Pierce Reacti-Bind™

Duplicates of E. coli, B. cepacia, B. subtilis, and S. epidermidis were transferred to individual wells of a Reacti-Bind™ Streptavidin Coated High Binding Capacity Clear 8-well Strips with SuperBlock® Blocking Buffer and incubated 30 minutes at room temperature to allow hybridization and capture to the well. Each well was then washed with 250 μl of wash buffer [100 mM Tris, 150 mM sodium chloride, 0.05% Tween® 20 (v/v), 0.01% sodium azide, 0.1% Zonyl® FSA (v/v), pH 7.2]. The well contents were then discarded with a flicking motion to remove essentially all of the liquid. The washing process was repeated five times with 250 μl of wash buffer for each wash. After discarding the final wash, 200 μl of AP Substrate was added to each well. The substrate was incubated in each well for 20 minutes at room temperature while protected from light with aluminum foil. At the completion of the substrate incubation, the 200 μl of substrate was transferred into a Celsis Advance™ cuvette to be placed in the Celsis Advance™ Luminometer instrument.

### Streptavidin Coated Cuvette

Duplicates of E. coli, B. cepacia, B. subtilis, and S. epidermidis were transferred to individual streptavidin coated cuvette and incubated 30 minutes at room temperature to allow hybridization and capture to the cuvette. Each well was then washed with 2.0 ml of wash buffer (100 mM Tris, 150 mM sodium chloride, 0.05% Tween® 20 (v/v), 0.01% sodium azide, 0.1% Zonyl® FSA (v/v), pH 7.2) by capping the cuvette and inverting to mix. The cuvette contents were then discarded with a flicking motion to remove essentially all of the liquid; the washing process was repeated five times with 2.0 ml of wash buffer for each wash. After the final wash the cuvettes were turned upside down on a paper towel and blotted. Then 200 μl of AP Substrate was added to each cuvette. The substrate was incubated in each cuvette for 20 minutes at room temperature and protected from light with aluminum foil.

### Results

The cuvettes for both the Pierce: wells and the Celsis cuvettes were placed in the Celsis Advance™ Luminometer instrument. The instrument was operated with the following parameters: no injectors, 10 second read, zero cal cutoff, and 0.6 cal factor for RLU.

TABLE 4
Results of Comparison Between Pierce Reacti-Bind ™ and
Streptavidin Coated Cuvettes

| Sample ID | RLU1 | RLU2 | AVE RLU | RATIO | Read Date |
|---|---|---|---|---|---|
| 1 X AP Substrate Only | 113 | 136 | 125 | NA | Nov. 7, 2007 |
| E. coli cuv | 861592 | 821155 | 841374 | 464.3 | Nov. 7, 2007 |
| B. cepacia cuv | 33391 | 30184 | 31788 | 17.5 | Nov. 7, 2007 |
| B. subtilis cuv | 4818 | 1388 | 3103 | 1.7 | Nov. 7, 2007 |
| S. epidermidis cuv | 1211 | 1271 | 1241 | 0.7 | Nov. 7, 2007 |
| Negative Control cuv | 2873 | 750 | 1812 | 1.0 | Nov. 7, 2007 |
| Positive Control cuv | 2430116 | 2407562 | 2418839 | 1334.9 | Nov. 7, 2007 |
| E. coli Pierce | 31786 | 28155 | 29971 | 54.0 | Nov. 7, 2007 |
| B. cepacia Pierce | 2496 | 2310 | 2403 | 4.3 | Nov. 7, 2007 |
| B. subtilis Pierce | 542 | 534 | 538 | 1.0 | Nov. 7, 2007 |
| S. epidermidis Pierce | 690 | 644 | 667 | 1.2 | Nov. 7, 2007 |
| Negative Control Pierce | 582 | 527 | 555 | 1.0 | Nov. 7, 2007 |
| Positive Control Pierce | 206430 | 216630 | 211530 | 381.1 | Nov. 7, 2007 |

The streptavidin coated cuvettes resulted in an increase in RLU signal for the Gram-negative organisms and an increase in the ratios of Gram-negative organism RLU signal over the negative control RLU signal, when compared to the Pierce Reacti-Bind™ Streptavidin coated wells using the same method for calculating the ratios. The ratio of E. coli increased 8.6 times in the Celsis cuvette, when compared to the ratio the Pierce wells. The ratio for the B. cepacia increased 4.0 times. The Gram-positive organisms of B. subtilis and S. epidermidis tested negative, less than 2 times the average negative control RLU, for both capture methods.

### Example 7

### 42° C. vs. Room Temperature Capture in Streptavidin Coated Cuvettes

The purpose of this example was to assess the difference in assay signal by comparing the results from increasing the capture temperature from ambient to 42° C.

Nucleic acid isolates stored at −80° C. of E. coli and B. subtilis were prepared according to in Example 1 and were used for analysis.

A hybridization mix was assembled for each hybridization reaction with the following components: 75 μl hybridization buffer [200 mM MOPS, 1 mM magnesium chloride, 3 M sodium chloride, 0.05% Tween 20 (v/v), 0.01% sodium azide, 0.2% Zonyl® FSA (v/v), pH 6.9]; RNase A-RPA Grade at 25 ng/75 μl); 5 μl probe 1 and probe 2 mixture (probe 1 and probe 2 (Table 2) SEQ ID NO.: 1 and SEQ ID NO.: 2, respectively, had been combined at a concentration of 1 pmole each/μl); and 2 μl probe 3 ((Table 2) SEQ ID NO.: 3) diluted to 1.25 pmoles/μl with AP preservation buffer (3 M sodium chloride, 30 mM Tris, 1 mM magnesium chloride, 0.1 mM zinc chloride, 0.05% sodium azide).

### Hybridization

All reactions were performed in duplicate, except for the E. coli reaction which had four replicates. For each hybridization reaction, 82 μL of the hybridization mix was placed in a polypropylene cuvette, along with 75 μl of isolated sample nucleic acids. Two controls were also assayed: the negative control consisted of 75 μl of water, and the positive control consisted of 1 μl of probe 4 (SEQ ID NO.: 4) along with 75 μl of water. The polypropylene cuvettes containing hybridization mix and sample or control were placed on an modular block dry-bath incubator at 42° C. for 30 minutes. The cuvettes were then removed from the dry-bath incubator, and the samples were transferred to a streptavidin coated cuvette. Two of the E. coli samples were incubated at room temperature for 30 minutes; the other reactions were incubated 30 minutes at 42° C. in the dry-bath incubator to allow hybridization and capture to the cuvette. Each well was then washed with 1.0 ml of wash buffer [100 mM Tris, 150 mM sodium chloride; 0.05% Tween® 20 (v/v), 0.01% sodium azide, and 0.1% Zonyl® FSA (v/v), pH 7.2] and vortexed for 10 seconds for each wash. The cuvette contents were then discarded with a flicking motion to remove essentially all of the liquid; the washing process was repeated three times with 1.0 ml of wash buffer for each wash. Then 200 μl of AP Substrate was added to each cuvette. The substrate was incubated in each cuvette for 20 minutes at room temperature and protected from light with aluminum foil.

### Results

The Celsis cuvettes were placed in the Celsis Advance™ Luminometer instrument. The instrument was operated with the following parameters: no injectors, 10 second read, zero cal cutoff, and 0.6 cal factor for RLU.

TABLE 5
RLU Results of 42° C. Versus Room Temperature Capture

| Sample ID | Result | RLU1 | RLU2 | RLU | Read Date |
|---|---|---|---|---|---|
| AP Substrate only | Negative | 127 | 119 | 123 | Nov. 20, 2007 |
| E. coli RT | Positive | 499768 | 476363 | 488066 | Nov. 20, 2007 |
| E. coli 42 | Positive | 646417 | 670797 | 658607 | Nov. 20, 2007 |
| B. subtilis 42 | Negative | 554 | 454 | 504 | Nov. 20, 2007 |
| Negative Control 42 | Positive | 585 | 1535 | 1060 | Nov. 20, 2007 |
| Positive Control 42 | Positive | 2569863 | 2381859 | 2475861 | Nov. 20, 2007 |

Surprisingly, the E. coli samples that were captured at 42° C. had an RLU that was 35% higher than the RLU for the samples that were captured at room temperature.

### Example 8

### Hybridization to Model Distinction of Gram-Negative from Gram-Positive Organisms

The following experiment was performed using synthetic single-stranded DNA as a template to model denatured or single-stranded DNA. The five DNA templates were designed to represent the target region of the following genera of bacteria: Bacillus, Staphylococcus, Streptococcus, and Listeria along with a synthetic DNA oligomer to represent the target region of most Enterobacteriaceae Gram-negative organisms.

A hybridization mix was assembled with the following components per reaction: 5 μl probe 1 (SEQ ID NO.: 1); 2 μl of a 1:100 dilution of the AP signal probe 5′-CGG TGC TTC TTC TGC GTT TTT TTT TT-3′ (SEQ ID NO.: 3) conjugated with alkaline phosphatase at 250 pmoles/μl in AP preservation buffer (3 M sodium chloride, 30 mM Tris, 1 mM magnesium chloride, 0.1 mM zinc chloride, 0.05% sodium azide) diluted in AP dilution buffer (100 mM Tris, 100 mM sodium chloride, 5 mM magnesium chloride, 0.01% sodium azide, pH 9.0); 1 μl of RNase A at 250 ng/μL; 75 μl Hybridization Buffer [200 mM MOPS, 3 M sodium chloride, 0.05% Tween 20 (v/v), 0.01% sodium azide, pH 6.9, 0.1% Zonyl® FSA]; and 66 μl of sterile DI water bringing the hybridization mix volume up to 149 μl. Hybridization mix was added to 24 wells of Pierce Reacti-Bind™ Streptavidin Coated High Binding Capacity Clear 8-well Strips with SuperBlock® Blocking Buffer (149 μl hybridization mix per well). The five synthetic DNA targets and one negative control were added to the wells with 4 reactions per target and control; 1 μl of each target at 0.5 pmole/μl was used per reaction. Sterile DI water (1 μl) was used for a negative control. Twelve wells (duplicates of each target and the negative control) were placed in a water bath at 31° C. for 30 minutes (Table 6-Water). The other twelve wells (duplicates of each target and the negative control) were placed into a 31° C. incubator (Model No. 120, Lab-Line Instruments Inc., Melrose Park, Ill.) for 30 minutes (Table 6-Inc). The wells were then washed with 250 μl of wash buffer [100 mM Tris, 150 mM sodium chloride, 0.05% Tween 20 (v/v), 0.01% sodium azide, 0.1% Zonyl® (v/v), pH 7.2]. The wash buffer was then discarded with a flicking motion to remove essentially all of the liquid; the washing process was repeated five times with 250 μl of wash buffer for each wash. After discarding the final wash, 200 μl Attoglow™ AP Substrate—450^{LB} diluted 1:10 with AP dilution buffer was added to each well and incubated for 20 minutes at room temperature and protected from light with aluminum foil. At the completion of the substrate incubation, the 200 μl of substrate was transferred with a pipette into Celsis Advance™ cuvettes. The cuvettes were placed in the Celsis Advance™ Luminometer instrument. The instrument was operated with the following parameters: no injectors were used, 10 second read, zero cal cutoff, and 0.6 cal factor for RLU. The substrate had excess background, so the wells were washed two more times with 250 μl of wash buffer, and another substrate was used. For the second read, 200 μl of CSPD® substrate was added to each well. The substrate incubated for 20 minutes at room temperature protected from light with aluminum foil. At the completion of the substrate incubation, the 200 μl of substrate was transferred with a pipette into Celsis Advance™ cuvettes. The cuvettes were placed in the Celsis Advance™ Luminometer instrument. The instrument was operated with the following parameters: no injectors were used, 10 second read, zero cal cutoff, and 0.6 cal factor for RLU. The probes show specificity for the target region of Gram-negative organisms over Gram-positive organisms using representative DNA segments.

TABLE 6
Results of Assay with Synthetic Single-Stranded DNA Targets.

| Sample ID | RLU1 | RLU2 | AVG RLU |
|---|---|---|---|
| Roche substrate | 105 | 110 | 108 |
| Inc- Gram-negative | 41532 | 33310 | 37421 |
| Inc- Bacillus | 224 | 175 | 200 |
| Inc- Streptococcus | 126 | 193 | 160 |
| Inc- Staphylococcus | 146 | 137 | 142 |
| Inc- Listeria | 154 | 154 | 154 |
| Inc- Negative | 124 | 122 | 123 |
| Water- Gram-negative | 39517 | 43247 | 41382 |
| Water- Bacillus | 276 | 208 | 242 |
| Water- Streptococcus | 118 | 134 | 126 |
| Water- Staphylococcus | 142 | 158 | 150 |
| Water- Listeria | 175 | 157 | 166 |
| Water- Negative | 121 | 121 | 121 |

Inc - 31° C. incubator; Water - 31° C. Water Bath

TABLE 7

Sequences of Synthetic Single-Stranded
DNA Targets.

| | |
|---|---|
| NAME | SEQUENCE |

| | |
|---|---|
| GN 504_560 SENSE | GTTACCCGCAGAAGAAGCACCGGCTAACTCCG |
| POS CTRL | TGCCAGCAGCCGCGGTAATACGG |
| | (SEQ ID NO.: 7) |
| SUBTILIS 504 | GGTACCTAACCAGAAAGCCACGGCTAACTACG |
| SENSE CTRL | TGCCAGCAGCCGCGGTAATACGT |
| | (SEQ ID NO.: 8) |
| STAPH 504 SENSE | GGTACCTAATCAGAAAGCCACGGCTAACTACG |
| CTRL | TGCCAGCAGCCGCGGTAATACGT |
| | (SEQ ID NO.: 9) |
| STREP 504 SENSE | GGTAGCTTACCAGAAAGGGACGGCTAACTACG |
| CTRL | TGCCAGCAGCCGCGGTAATACGT |
| | (SEQ ID NO.: 10) |
| LISTERIA 504 | GGTATCTAACCAGAAAGCCACGGCTAACTACG |
| SENSE CTRL | TGCCAGCAGCCGCGGTAATACGT |
| | (SEQ ID NO.: 11) |

### Example 9

### Species Specific Cultures for Nucleic Acid Isolation

The following species specific cultures as described in Table 1 and in Example One above were propagated for nucleic acid isolation in Letheen media: E. coli, B. subtilis, B. cepacia, S. epidermidis, B. cercus, R. pickettii, E. gallinarum, C. albicans, S. cerevisiae, P. aeruginosa, P. fliiorescens, P. putida, E. faecium, K. rhizophila and S. maltophilia.

### Lysis of Overnight Cultures

Cells of each of the above cultures were pelleted by aliquoting 3 ml of each overnight culture into individual sterile 3.5 ml 55 mm×12 mm polystyrene tubes (Sarstedt) and centrifuging at 2,000×g for 3 minutes. The supernatant was decanted and discarded. Five hundred microliters of lysis/extraction buffer was added to each pellet and then gently vortexed to resuspend each pellet and then transferred to a corresponding 1.5 mL skirted conical tube (Simport) containing 0.5 g of dry 0.5 mm zirconia/silica beads (BioSpec Products; Bartlesville, Okla.). The lysis/extraction buffer used above was composed of 100 mM 3-(N-morpholino)-propanesulfonic acid (MOPS), 10 mM ethylenediaminetetraacetic acid (EDTA), 1% SDS, 5 mM dithiothreitol (DTT), 0.5% of Antifoam A (Sigma Aldrich) exemplary of a silicone polymer based antifoam 0-5% of a 30% active silicone emulsion Antifoam Y-30 (Sigma Aldrich) exemplary of a water disbursable antifoam designed to control foam in aqueous systems and 50 μM aurintricarboxylic acid (or its salts, such as the ammonium salt), sold as Alumion™ (Sigma Aldrich) at a final pH 7.0 for the combined reagents.

The lysis tubes were then placed on a Deluxe PulseVortex Mixer (Fisher Scientific) and vortexed on pulse setting, 3000×rpm for 30 seconds and resting for 10 seconds programmed to repeat this process over the course of 10 minutes. Following lysis, the tubes containing the lysate were set aside for desalting.

### Desalting and Filtration

Desalting spin columns (BioVentures, Inc.) were prepared for storage buffer removal by twisting off the bottom tab, turning the top screw cap one-quarter turn and then placing each of the columns into a collection tube consisting of a Celsis Advance™ Cuvette (55 mm×12 mm polystyrene tube) and centrifuging each column-tube assembly at 1,000×g for 1 minute. Collection tubes were discarded as waste and each of the centrifuged spin columns was then transferred to a fresh Celsis Advance™ Cuvette to form a spin column-tube assembly.

The cell lysates obtained by pulse vortexing were then desalted using 100 μL of each lysate by transfer to an individual and corresponding prepared spin column-tube assembly with each lysate processed in duplicate. Each assembly with applied lysate was then centrifuged at 1000×g for 2 minutes and the column filtrate was collected in the tube portion of the assembly. Each applied lysate yielded ˜100 μL of filtrate in its respective collection tube. Each lysate was subsequently evaluated for nucleic acids by gel electrophoresis and 75 μL of each filtrate was Used in the hybridization reactions and detection reactions described below.

### Gel Electrophoresis

Gel electrophoresis was used to confirm the presence of rRNA in the collected 100 μL column filtrate for each sample from above. Samples were prepared for electrophoresis on a 1.2% agarose gel, such as the FlashGel® RNA Cassette (Lonza, Rockland, Me. US), using a 4 μL aliquot of the filtrate and 1 μL Formaldehyde Sample Buffer (Lonza). One microliter of FlashGel® RNA Marker, which contained RNA fragments in sizes from 0.5 to 9 kb (0.5, 1, 1.5, 2, 2.5, 3, 4, 5, 6, and 9 kb) (Lonza), and 4 μL Formaldehyde Sample Buffer was used as a size marker. The prepared samples were denatured at 65° C. for 5 minutes and then loaded into individual wells on a FlashGel® RNA Cassette and electrophoresed at 225 volts for 8 minutes. The FlashGel® RNA Cassette was stained for approximately 20 minutes following the gel manufacturer's suggested protocol. The FlashGel® RNA Cassette was placed on a high performance ultraviolet transilluminator, and the image was captured with a Kodak Gel Logic 100 camera (Kodak, Rochester, N.Y., USA).

### Electrophoretic Analysis Results

A positive result was indicated by the observation and appearance of rRNA bands of appropriate size and relative intensities, as well as the presence of genomic DNA, in the gel photographs. The electrophoretic results indicated that the methods used for culture, lysis, and nucleic acid isolation yielded adequate amounts of rRNA and genomic DNA of sufficient quality for analysis.

### Detection Assay

The isolated nucleic acids from the species specific cultures described above were assayed by the methods set forth in this disclosure consisting of hybridization and solid phase capture with, a set of probes designed to preferentially distinguish and detect 16S rRNA of Gram-negative organisms from other microbial organisms.

### Hybridization and Capture

For each hybridization reaction, 75 μl of each filtrate from the lysed and spin-column cleaned species specific culture set forth above was combined with 75 μl of hybridization buffer in a hybridization tube containing two lyophilized probes (Celsis) present at 5 pmoles each (based on A260). One of the probes consisted of a biotinylated DNA oligonucleotide capture probe and the other probe consisted of an alkaline phosphatase labeled DNA oligonucleotide signal probe that were both designed to hybridize to a segment of the 16S rRNA of Gram-negative organisms consistent with the description in this disclosure. The hybridization buffer was comprised of 200 mM MOPS, 3 M sodium chloride, 0.05% Tween® 20 (v/v), 0.01% sodium azide, 0.2% Zonyl® FSA (anionic lithium carboxylate fluorosurfactant) (v/v), 1 mM magnesium chloride, 25 ng RNase A (Worthington Biochemical; Lakewood, N.J. US) at a final pH of 7.5. The RNase used above was from a stock solution consisting of 1 mg/ml RNase A dissolved in 10 mM HEPES, 20 mM sodium chloride, 1 mM EDTA, 0.1% Triton-X, 50% glycerol, pH 6.9 The two probes for detecting Gram-negative organisms utilized in this example were of different sequence than the capture and signal probes listed in Table 2. A negative control, which consisted of 75 μl of 1 mM sodium citrate buffer, pH6.4, and a positive control were utilized to monitor the assay performance. The positive control consisted of 75 μl of 1 mM sodium citrate buffer, pH6.4 and 2.5 pmoles of a DNA oligonucleotide that exactly corresponded to the segment of 16S rRNA in Gram-negative organisms complimentary to and target by the two lyophilized probes present in the hybridization tube. The hybridization tubes Containing hybridization buffer and culture lysate filtrates or controls were placed on an Isotemp® modular block dry-bath incubator (Fisher Scientific) at 42° C. for 30 minutes. Each hybridization tube was then removed from the dry-bath incubator, and the individual hybridization mixtures were transferred to corresponding individual streptavidin coated capture-cuvettes (Celsis), and the capture-cuvettes were then placed on a modular block dry-bath incubator at 42° C. for 30 minutes. Each of the incubated capture-cuvettes was then washed three times. Each wash was performed by 10 seconds of vortexing with 1 ml of wash buffer consisting of 100 mM Tris, 150 mM sodium chloride, 0.05% Tween® 20(v/v), 0.01% sodium azide, 0.1% Zonyl® FSA (v/v), pH 7.2. The final wash contents were then discarded with a flicking motion to remove substantially all of the wash liquid. Next, 200 μl of an alkaline phosphatase substrate, AP SUBSTRATE (Michigan Diagnostics, Royal Oak, Mi) was added to each cuvette. The cuvettes containing substrate were then promptly analyzed on a Celsis Advance™ Luminometer.

### Analysis and Results

The Celsis streptavidin cuvettes containing substrate were placed in the Celsis Advance™ Luminometer instrument for analysis. The instrument was operated with the following parameters: no injectors, 10 second read, zero cal cutoff, and 0.6 cal factor for RLU (Relative Light Units). The results obtained from the luminometer for the processed cell cultures and controls are tabulated and duplicate (RLU1 and RLU2) readings and their averages (AVG RLU) for each sample type appear in Table 8 below.

TABLE 8
Analysis Gram status of nucleic acids from 15 organisms

| Sample ID | Result | RLU1 | RLU2 | AVG RLU |
|---|---|---|---|---|
| Negative control | Negative | 746 | 650 | 698 |
| R. pickettii | Positive | 192112 | 342736 | 267424 |
| B. cereus | Negative | 1040 | 997 | 1019 |
| E. gallinarum | Negative | 695 | 647 | 671 |
| C. albicans | Negative | 820 | 854 | 837 |
| S. cerevisiae | Negative | 743 | 1203 | 973 |
| P. aeruginosa | Positive | 609805 | 884702 | 747254 |
| P. fluorescens | Positive | 658193 | 942273 | 800233 |
| P. putida | Positive | 1200965 | 947134 | 1074050 |
| E. faecium | Negative | 980 | 1147 | 1064 |
| K. rhizophila | Negative | 1632 | 1478 | 1555 |
| E. coli | Positive | 1173016 | 1567230 | 1370123 |
| S. maltophilia | Positive | 59480 | 58730 | 59105 |
| B. cepacia | Positive | 235707 | 263621 | 249664 |
| B. subtilis | Negative | 1861 | 2410 | 2136 |
| S. epidermidis | Negative | 3224 | 2882 | 3053 |
| Positive control | Positive | 2155546 | 2302713 | 2229130 |

The cutoff utilized for a negative result was set as 5000 RLU or lower and a positive detection of a Gram-negative organism was considered 5001 RLU or higher. All of the species specific cultures that were inoculated with Gram-negative organisms had an average RLU that was greater than 5001 RLU, thus giving a positive result. All of the species specific cultures inoculated with non-Gram-negative organisms had an average RLU that was less than 5000 RLU, thus giving a negative result. Only lysates obtained from Gram-negative organisms gave positive findings.

### Evaluation of Lyophilization of Additional Probe Sets

A set of probes consisting of a biotinylated DNA oligonucleotide capture probe and an alkaline phosphatase labeled DNA oligonucleotide signal probe were designed to preferentially distinguish and detect 16S rRNA of Staphylococcus aureus from other microbial organisms were lyophilized in polypropylene tubes (Celsis) using 5 pmoles of each probe per tube to provide a hybridization tube containing a lyophilized probe set for detection of S. aureus. Like wise hybridization tubes containing lyophilized probes were prepared using 5 pmoles of each probe from the respective probe sets for detection of Gram-negative proteobacteria organisms or fungi, as described above, to provide hybridization tubes for the detection of Gram-negative proteobacteria or fungi respectively.

Lysates of E. coli. B, subtilis, B. cepacia, S. epidermidis, B. cereus, R. pickettii, E. gallinarum, C. albicans, S. cerevisiae, P. aeruginosa, P. fluorescens, P. putida, E. faecium, K. rhizophila and S. maltophilia were prepared by following the steps outlined in the lysis of overnight cultures and desalting filtration sections described in Example 9. Reactions and analyses were performed by the method described in Example 9 using the lysates above, a negative control and positive controls as in Example 9 above for Gram-negative proteobacteria, S. aureus and fungi.

Distinction of the S. aureus positive control from the analyzed organisms was achieved by the S. aureus positive control having an RLU greater than 5001 and all of the organisms having a negative result with an RLU of 5000 of less. Subsequently, S. aureus 16S rRNA nucleic acid isolated as essentially set forth by the methods in Example 9 was found to have a positive result with a RLU greater than 5001.

Distinction of C. albicans and S. cerevisiae from the other analyzed organisms was achieved by C. albicans and S. cerevisiae having an RLU greater than 5001 and all of the other organisms having a negative result with an RLU of 5000 or less.

Results indicate that the probe sets can be lyophilized and subsequently used in reactions without compromising their functionality and utility in detecting their respective organisms without introducing cross reactivity or non-specific binding.

### Example 10

An experiment was conducted to determine if by using essentially the method described in Example 9, probes designed to hybridize to 16S rRNA of Gram-negative organisms, probes designed to hybridize 16S rRNA of S. aureus, and probes designed to hybridize to 18S rRNA of fungi, could be combined in one hybridization reaction permitting the selective determination of the target organism without the probes interfering with one another or creating spurious non-specific and interfering signal.

### Lysis and Filtration

Lysates of E. coli, S. epidermidis, S. aureus and C. albicans were prepared by essentially following the steps outlined in the lysis of overnight, cultures and desalting filtration sections described in Example 9.

### Three Probe Set Combination Reactions

Reactions containing a combination of three different probe sets were assembled in duplicate in a polypropylene tube for each lysate, a negative control, and a positive control. The reactions consisted of 75 μl of hybridization buffer as described in Example 9 containing, 5 pmoles of a biotinylated DNA oligonucleotide capture probe and 5 pmoles of an alkaline phosphatase labeled DNA oligonucleotide signal probe designed to hybridize to the 16S rRNA of Gram-negative organisms, 5 pmoles of a biotinylated DNA oligonucleotide capture probe and 5 pmoles of an alkaline phosphatase labeled DNA oligonucleotide signal probe designed to hybridize to the 16S rRNA of S. aureus, 5 pmoles of a biotinylated DNA oligonucleotide capture probe and 5 pmoles of an alkaline phosphatase labeled DNA oligonucleotide signal probe, designed to hybridize to a homologous segment of the 18S rRNA common to C. albicans, A. niger and S. cerevisiae or similar fungi. For each lysate reaction, 75 μl of lysate was added to a hybridization tube containing the hybridization buffer and three probe sets. The negative controls consisted of 75 μl of 1 mM sodium citrate buffer, pH6.4. The positive controls consisted of essentially 75 μl of 1 mM sodium citrate buffer, pH6.4 and three synthetic DNA oligonucleotide targets consisting of 0.5 pmoles each of a DNA oligonucleotide that exactly corresponded to the segment of 16S rRNA in Gram-negative organisms complimentary to and target by the capture probe and signal probe designed to hybridize to 16S rRNA of Gram-negative proteobacteria organisms (hereafter denoted as Gram-negative), a DNA oligonucleotide that exactly corresponded to the segment of 16S rRNA in S. aureus complimentary to and targeted by the capture probe and signal probe designed to hybridize to 16S rRNA of S. aureus, and a DNA oligonucleotide that exactly corresponded to a homologous segment of the 18S rRNA common to C. albicans, A. niger and S. cerevisiae or similar fungi (hereafter denoted as fungi).

### Individual Probe Set Reactions

Reactions containing individual probe sets used in the foregoing three probe set combination targeting either Gram-negative proteobacteria organisms, S. aureus or fungi were each prepared in duplicate in polypropylene tubes using 75 μl hybridization buffer containing 5 pmoles of the biotinylated capture probe and 5 pmoles of the alkaline phosphatase labeled signal probe and 75 μl of lysate from a culture of the respective corresponding target organisms as shown in Table 9 below.

### Hybridization and Capture

The hybridization tubes containing hybridization buffer, probes, and lysates or controls were incubated, washed, and prepared for analysis by the method described in

### Example 11

### Results

TABLE 9

| Sample_ID | Probes | Result | RLU1 | RLU2 | AVG RLU |
|---|---|---|---|---|---|
| negative control | Gram-negative, S. aureus, fungi | Negative | 1045 | 1084 | 1065 |
| C. albicans | fungi | Positive | 74877 | 80543 | 77710 |
| C. albicans | Gram-negative, S. aureus, fungi | Positive | 85814 | 94358 | 90086 |
| S. aureus | S. aureus | Positive | 44921 | 45731 | 45326 |
| S. aureus | Gram-negative, S. aureus, fungi | Positive | 45422 | 54401 | 49912 |
| E. coli | Gram negative | Positive | 1521405 | 1362559 | 1441982 |
| E. coli | Gram-negative, S. aureus, fungi | Positive | 1179717 | 1278683 | 1229200 |
| S. epidermidis | Gram-negative, S. aureus, fungi | Negative | 3905 | 2970 | 3438 |
| positive control | Gram-negative, S. aureus, fungi | Positive | 1730593 | 1641460 | 1686027 |

The cutoff utilized for a negative result was set as 5000 RLU or lower and a positive detection of a Gram-negative proteobacteria organism, S. aureus, or a fungi was considered 5001 RLU or higher. The Gram-positive organism, S. epidermidis, had a negative RLU when all three probe sets were combined in the same reaction. E. coli, S. aureus, and C. albicans all maintained a positive RLU when the three probe sets were, combined. The data showed that probes designed to hybridize to the 16S rRNA of Gram-negative proteobacteria organisms or S. aureus, or the 18S rRNA of fungi could be combined in the same reaction and maintain specificity for their respective targets without any significant increase in background signal.

While various embodiments of the present invention have been described above, it should be understood that such disclosures have been presented by way of example only, and are not limiting. Thus, the breadth and scope of the present invention should not be limited by any of the above-described exemplary embodiments, but should be defined only in accordance with the following claims and their equivalents.

Having now fully described the invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference in their entirety.

1. A method of detecting the presence of at least one nucleotide sequence of interest in a sample comprising:
a) providing a sample potentially containing at least one nucleotide sequence of interest;
b) creating a mixture by combining:
i) the sample,
ii) at least one nucleic acid probe labeled with a detectable label, and
iii) a nuclease capable of degrading the sequence of interest;
wherein the nuclease is added to the sample before or concurrently with adding the probe;
wherein a complex forms between the sequence of interest and the probe; and
c) measuring the level of the detectable label in the complex, wherein the presence of the detectable label in the complex indicates the presence of the sequence of interest.

2. A method of detecting the presence of at least one nucleotide sequence of interest in a sample comprising:
a) providing a sample potentially containing at least one nucleotide sequence of interest;
b) creating a mixture by combining:
i) the sample, and
ii) a combination of at least one nucleic acid probe labeled with a detectable label, and a nuclease capable of degrading the sequence of interest; wherein a complex forms between the sequence of interest and the probe; and
c) measuring the level of the detectable label in the complex, wherein the presence of the detectable label in the complex indicates the presence of the sequence of interest.

3. A method of detecting the presence of at least one nucleotide sequence of interest in a sample comprising:
a) providing a sample potentially containing at least one nucleotide sequence of interest;
b) creating a mixture by combining:
i) the sample,
ii) at least one nucleic acid probe labeled with a detectable label, and
iii) a nuclease capable of degrading the sequence of interest;
wherein the probe is added to the sample within a selected time period;
wherein a complex forms between the sequence of interest and the probe; and
c) measuring the level of the detectable label in the complex, wherein the presence of the detectable label in the complex indicates the presence of the sequence of interest.

4. The method according to claim 3, wherein the selected time period is from about 0 to about 15 minutes after addition of the nuclease.

5. A method of detecting the presence of at least one nucleotide sequence of interest in a sample comprising:
a) providing a sample potentially containing at least one nucleotide sequence of interest;
b) creating a mixture by combining:
i) the sample,
ii) at least one nucleic acid probe labeled with a detectable label, and
iii) a nuclease capable of degrading the sequence of interest;
wherein the nuclease is added to the sample and the probe before the sequence of interest hybridizes to the probe, resulting in a selected percentage of hybridization;
wherein a complex forms between the sequence of interest and the probe; and
c) measuring the level of the detectable label in the complex, wherein the presence of the detectable label in the complex indicates the presence of the sequence of interest.

6. The method of claim 5, wherein the selected percentage of hybridization is from about 5% to about 95%.

7. A method of detecting the presence of at least one nucleotide sequence of interest in a sample comprising:
a) providing a sample potentially containing at least one nucleotide sequence of interest;
b) creating a mixture by combining:
i) the sample,
ii) at least one nucleic acid probe labeled with a detectable label, and
iii) a nuclease capable of degrading the sequence of interest; and
iv) at least one fluorosurfactant;
wherein a complex forms between the sequence of interest and the probe; and
c) measuring the level of the detectable label in the complex, wherein the presence of the detectable label in the complex indicates the presence of the sequence of interest.

8. The method according to claim 7, wherein the fluorosurfactant is selected from the group of an anionic fluorosurfactants, cationic fluorosurfactants, amphoteric fluorosurfactants, nonionic fluorosurfactants, zwitterionic fluorosurfactants, and mixtures thereof.

9. The method according to any of claims 1, 2, 3, or 5, further comprising adding at least one fluorosurfactant to the mixture.

10. The method according to claim 9, wherein the at least one fluorosurfactant is selected from the group consisting of anionic fluorosurfactants, cationic fluorosurfactants, amphoteric fluorosurfactants, nonionic fluorosurfactants, zwitterionic fluorosurfactants, and mixtures thereof.

11. The method according to any of claims 1, 2, 3, 5, or 7, further comprising extracting the nucleotide sequence of interest from the sample before creating the mixture.

12. The method according to any of claims 1, 2, 3, 5, or 7, wherein the nuclease is selected from the group consisting of RNase A, RNase T1, RNase I, and S1 nuclease.

13. The method according to any of claims 1, 2, 3, 5, or 7, wherein the complex is immobilized.

14. The method according to claim 13, further comprising washing the complex with a wash buffer.

15. The method according to any of claims 1, 2, 3, 5, or 7, further comprising adding a reagent substrate to the complex.

16. The method according to any of claims 1, 2, 3, 5, or 7, wherein the probe comprises a capture probe labeled with biotin and a signal probe labeled with alkaline phosphatase.

17. The method according to claim 16, further comprises adding a reagent substrate to the complex, wherein the reagent substrate is selected from the group consisting of adamantyl-1,2-dioxetane phosphate, 5-bromo-4-chloro-3-indolyl phosphate/nitro blue tetrazolium and para-nitrophenyl phosphate.

18. A kit comprising:
a) at least one nucleic acid probe labeled with a detectable label;
b) at least one fluorosurfactant; and
c) a nuclease capable of degrading a nucleotide sequence of interest.

19. The kit of claim 18, further comprising instructions for using the kit.

20. The kit of claim 18, further comprising a substrate reagent.

21. A method of detecting the presence of a target of interest in a sample comprising:
a) providing a sample potentially containing a target of interest;
b) creating a mixture by combining:
i) the sample;
ii) at least one probe labeled with a detectable label; and
iii) at least one fluorosurfactant;
wherein a complex forms between the target of interest and the probe; and
c) measuring the level of the detectable label in the complex, wherein the presence of the detectable label in the complex indicates the presence of the target of interest.

22. The method of claim 21, wherein the at least one fluorosurfactant is selected from the group consisting of anionic fluorosurfactants, cationic fluorosurfactants, amphoteric fluorosurfactants, nonionic fluorosurfactants, zwitterionic fluorosurfactants, and mixtures thereof.

23. The method of claim 21, wherein the target of interest is selected from the group consisting of proteins, peptides, small chemical molecules, carbohydrates, lipopolysaccharides, polysaccharides, and lipids.

24. A method of reducing non-specific binding of cells, subcellular organelles, biomolecules, or chemical molecules comprising:
a) adding at least one fluorosurfactant to a buffer; and
b) contacting the cells, subcellular organelles, biomolecules or chemical molecules with the buffer;
wherein the presence of the at least one fluorosurfactant results in the reduction of non-specific binding of the cells, subcellular organelles, biomolecules or chemical molecules to a surface or to each other.

25. The method of claim 24, wherein the at least one fluorosurfactant is selected from the group consisting of anionic fluorosurfactants, cationic fluorosurfactants, amphoteric fluorosurfactants, nonionic fluorosurfactants, zwitterionic fluorosurfactants, and mixtures thereof.

26. The method of claim 24, wherein the application is selected from the group consisting of an immunoassay, a microfluidic assay, passivation of a vessel surface, and cell culture.


SEQUENCE LISTING (11 sequences)

SEQ ID NO: 1 - DNA, 26 bp, Artificial
  Probe
tttttttttttattaccgcggctgct

SEQ ID NO: 2 - DNA, 14 bp, Artificial
  Probe
ggcacggagttagc

SEQ ID NO: 3 - DNA, 26 bp, Artificial
  Probe
cggtgcttcttctgcgtttttttttt

SEQ ID NO: 4 - DNA, 55 bp, Artificial
  Probe
gttacccgcagaagaagcaccggctaactccgtgccagcagccgcggtaatacgg

SEQ ID NO: 5 - DNA, 19 bp, Artificial
  Primer
ccgcagaagaagcaccggc

SEQ ID NO: 6 - DNA, 16 bp, Artificial
  Primer
tgtrtgaagaaggyct

SEQ ID NO: 7 - DNA, 55 bp, Artificial; identical to SEQ ID NO: 4
  Target

SEQ ID NO: 8 - DNA, 55 bp, Artificial
  Target
ggtacctaaccagaaagccacggctaactacgtgccagcagccgcggtaatacgt

SEQ ID NO: 9 - DNA, 55 bp, Artificial
  Target
ggtacctaatcagaaagccacggctaactacgtgccagcagccgcggtaatacgt

SEQ ID NO: 10 - DNA, 55 bp, Artificial
  Target
ggtagcttaccagaaagggacggctaactacgtgccagcagccgcggtaatacgt

SEQ ID NO: 11 - DNA, 55 bp, Artificial
  Target
ggtatctaaccagaaagccacggctaactacgtgccagcagccgcggtaatacgt